(19) United States
(12) Patent Application Publication
(10) Pub. No.: US 2024/0371461 A1
(43) Pub. Date: Nov. 7, 2024

(54) COMPUTATIONAL ARCHITECTURE FOR ENZYME DESIGN

(71) Applicant: University of Southern California, Los Angeles, CA (US)

(72) Inventors: Wenjun Xie, Cambridge, MA (US); Arieh Warshel, Los Angeles, CA (US)

(21) Appl. No.: 18/683,379

(22) PCT Filed: Jun. 15, 2022
(86) PCT No.: PCT/US2022/033608

§ 371 Date: Feb. 13, 2024

Related U.S. Application Data
(60) Provisional application No. 63/234,099, filed on Aug. 17, 2021.

(51) Int. Cl. G16B 15/20; G16B 20/50

(52) U.S. Cl. CPC G16B 15/20; G16B 20/50

(57) ABSTRACT

Aspects of this technical solution can include generating, by processors, a generative model based on homologous sequences of wild-type enzymes, determining, by the processors, whether a distance between a mutated amino acid residue of each of a plurality of enzyme mutants and a corresponding substrate, when the substrate is bound to the mutant, meets a first threshold, selecting, based on a result of the determination with the first threshold, a first candidate set of enzyme mutants, calculating, by the processors based on the generative model, a statistical energy of each mutant of the first candidate set of enzyme mutants, and identifying, by the processors based on the calculated statistical energies, a first set of enzyme mutants among the first candidate set of enzyme mutants, such that the first set of enzyme mutants have higher efficiency than remaining ones of the first candidate set of enzyme mutants.

### CROSS-REFERENCE TO RELATED APPLICATIONS

This application is a National Stage Entry under 35 U.S.C. § 371 of International Application No. PCT/US2022/033608, filed Jun. 15, 2022, which claims the benefit of and priority under 35 U.S.C. § 119 to U.S. Provisional Patent Application No. 63/234,099, entitled “System and Method for Computational Enzyme Design based on Maximum Entropy,” filed Aug. 17, 2021, the entirety of which is incorporated by reference herein.

### FEDERALLY SPONSORED RESEARCH OR DEVELOPMENT

This disclosure was made with Government support under contract no. R35 GM122472 awarded by the National Institutes of Health (NIH) and contract no. MCB 1707167 awarded by the National Science Foundation (NSF). The Government has certain rights in this invention.

### TECHNICAL FIELD

This disclosure generally relates to computing systems, including but not limited to a computational architecture for enzyme design.

### INTRODUCTION

Enzymes are extraordinary catalysts that play vital roles in nearly all biochemical processes. Designing efficient enzymes could help in solving catastrophic threats to humankind, including the energy crisis, environmental pollution, and food shortage. However, the use of computational modeling for enzyme design is still not at the stage when it can guide sufficiently reliable enzyme design. Advances in computational modeling utilizing physics-based approaches have been slow, and machine learning strategies have not been limited in their effectiveness for predicting the catalytic power of enzymes. For example, many attempts have failed to focus on active regions or distinguish between active and inactive regions within the enzymes, and thus are unable to properly predict or grade efficacy of an enzyme.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a block diagram illustrating an example of an enzyme design system for training and/or using a machine learning model, according to some implementations;

FIGS. 2A-2C are diagrams depicting an example of a maximum entropy model, according to some implementations;

FIGS. 3A-3C are graphs showing comparisons of sequence statistics obtained from a maximum entropy model and natural multiple sequence alignment (MSA), according to some implementations;

FIGS. 4A-4F are diagrams depicting an example of Pearson correlation coefficient between a statistical energy and enzyme efficiency, according to some implementations;

FIGS. 5A-5D are diagrams depicting an example of enzyme design using a maximum entropy model, according to some implementations;

FIG. 6 is a flowchart illustrating an example methodology for identifying enzyme mutants with improved efficiency and/or improved stability, according to some implementations;

FIG. 7A and FIG. 7B are block diagrams depicting embodiments of computing devices useful in connection with the methods and systems described herein;

FIG. 8 is a flowchart illustrating a method of enzyme design, according to some implementations; and

FIG. 9 is a flowchart illustrating a method of enzyme design, according to some implementations.

The details of various embodiments of the methods and systems are set forth in the accompanying drawings and the description below.

### SUMMARY

Various embodiments disclosed herein are related to a method for producing enzyme mutants. The method may include generating, by one or more processors, a maximum entropy model based on homologous sequences of wild-type enzymes. The method may include obtaining, by the one or more processors, information on a plurality of enzyme mutants. The method may include determining, by the one or more processors, whether a distance between a mutated amino acid residue of each mutant of the plurality of enzyme mutants and a corresponding substrate when the substrate is bound to the mutant is less than or equal to a first threshold, and selecting, based on a result of the determination with the first threshold, a first candidate set of enzyme mutants. The method may include calculating, by the one or more processors based on the maximum entropy model, a statistical energy of each mutant of the first candidate set of enzyme mutants. The method may include identifying, by the one or more processors based on the calculated statistical energies, a first set of enzyme mutants among the first candidate set of enzyme mutants, such that the first set of enzyme mutants have higher efficiency than remaining ones of the first candidate set of enzyme mutants.

In some embodiments, in determining the first candidate set, in response to determining that the distance between the mutated amino acid residue of each mutant of the plurality of enzyme mutants and the corresponding substrate when the substrate is bound to the mutant is less than or equal to the first threshold, the one or more processors may add the mutated amino acid residue to the first candidate set.

In some embodiments, in determining the first set of enzyme mutants, the one or more processors may identify, as mutants with improved efficiency, one or more enzyme mutants with energies lower than an energy of wild-type enzymes.

In some embodiments, the one or more processors may determine whether there are any remaining residues of each mutant of the plurality of enzyme mutants. In response to determining that there are no remaining residues in the plurality of enzyme mutants, the one or more processors may sample residues contained in the first candidate set to obtain the corresponding mutants in the first candidate set.

In some embodiments, the one or more processors may determine whether the distance between the mutated amino acid residue of each mutant of the plurality of enzyme mutants and the corresponding substrate when the substrate is bound to the mutant is greater than a second threshold, and select, based on a result of the determination with the second threshold, a second candidate set of enzyme mutants. The one or more processors may calculate, based on the maximum entropy model, a statistical energy of each mutant of the second candidate set of enzyme mutants. The one or more processors may identify, based on the calculated statistical energies of the second candidate set of enzyme mutants, a second set of enzyme mutants among the second candidate set of enzyme mutants, such that the second set of enzyme mutants have higher stability than remaining ones of the second candidate set of enzyme mutants. The second threshold may be greater than the first threshold. In selecting the second candidate set, in response to determining that the distance between the mutated amino acid residue of each mutant of the plurality of enzyme mutants and the corresponding substrate when the substrate is bound to the mutant is greater than the second threshold, the one or more processors may add the mutated amino acid residue to the second candidate set. In selecting the second set of enzyme mutants, the one or more processors may identify, as mutants with improved stability, one or more enzyme mutants with energies thereof lower than an energy of wild-type enzymes. The one or more processors may sample residues contained in the second candidate set to obtain the corresponding mutants in the second candidate set.

In some embodiments, the method may include producing, based on one of the first set of enzyme mutants, a recombinant enzyme comprising at least one non-naturally occurring amino acid mutation.

Various embodiments disclosed herein are related to a recombinant enzyme that may include at least one non-naturally occurring amino acid mutation.

In some embodiments, the recombinant enzyme may include one of the first set of enzyme mutants.

Various embodiments disclosed herein are related to a system for producing enzyme mutants. The system may include one or more processors in communication with one or more data storage devices storing an enzyme database, a machine learning model, and training instances. The one or more processors may be configured to generate a maximum entropy model based on homologous sequences of wild-type enzymes. The one or more processors may be configured to obtain information on a plurality of enzyme mutants. The one or more processors may be configured to determine whether a distance between a mutated amino acid residue of each mutant of the plurality of enzyme mutants and a corresponding substrate when the substrate is bound to the mutant is less than or equal to a first threshold, and select, based on a result of the determination with the first threshold, a first candidate set of enzyme mutants. The one or more processors may be configured to calculate, based on the maximum entropy model, a statistical energy of each mutant of the first candidate set of enzyme mutants. The one or more processors may be configured to identify, based on the calculated statistical energies, a first set of enzyme mutants among the first candidate set of enzyme mutants.

In some embodiments, in determining the first candidate set, the one or more processors may be configured to: in response to determining that the distance between the mutated amino acid residue of each mutant of the plurality of enzyme mutants and the corresponding substrate when the substrate is bound to the mutant is less than or equal to the first threshold, add the mutated amino acid residue to the first candidate set.

In some embodiments, in determining the first set of enzyme mutants, the one or more processors may be configured to identify, as mutants with improved efficiency, one or more enzyme mutants with energies lower than an energy of wild-type enzymes.

In some embodiments, the one or more processors may be configured to determine whether there are any remaining residues of each mutant of the plurality of enzyme mutants. The one or more processors may be configured to: in response to determining that there are no remaining residues in the plurality of enzyme mutants, sample residues contained in the first candidate set to obtain the corresponding mutants in the first candidate set.

In some embodiments, the one or more processors may be configured to determine whether the distance between the mutated amino acid residue of each mutant of the plurality of enzyme mutants and the corresponding substrate when the substrate is bound to the mutant is greater than a second threshold, and select, based on a result of the determination with the second threshold, a second candidate set of enzyme mutants. The one or more processors may be configured to calculate, based on the maximum entropy model, a statistical energy of each mutant of the second candidate set of enzyme mutants. The one or more processors may be configured to identify, based on the calculated statistical energies of the second candidate set of enzyme mutants, a second set of enzyme mutants among the second candidate set of enzyme mutants. The second threshold may be greater than the first threshold. In selecting the second candidate set, the one or more processors may be configured to: in response to determining that the distance between the mutated amino acid residue of each mutant of the plurality of enzyme mutants and the corresponding substrate when the substrate is bound to the mutant is greater than the second threshold, add the mutated amino acid residue to the second candidate set.

In some embodiments, in selecting the second set of enzyme mutants, the one or more processors may be configured to identify, as mutants with improved stability, one or more enzyme mutants with energies thereof lower than an energy of wild-type enzymes. The one or more processors may be configured to sample residues contained in the second candidate set to obtain the corresponding mutants in the second candidate set.

### DETAILED DESCRIPTION

Aspects of this technical solution are described herein with reference to the figures, which are illustrative examples of this technical solution. The figures and examples below are not meant to limit the scope of this technical solution to the present implementations or to a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted to not obscure the present implementations. Terms in the specification and claims are ascribed no uncommon or special meaning unless explicitly set forth herein. Further, this technical solution and the present implementations encompass present and future known equivalents to the known components referred to herein by way of description, illustration, or example.

Although the present technology discussed herein is discussed in the context of application to enzymes and enzyme design, the methods described herein can also be applied to other proteins or biomolecular entities. Non-limiting examples of such other entities include antibodies, protein receptors, hormones, growth factors, anticoagulation factors, transcription factors, antigens, and cytokines.

Enzymes are biological catalysts that speed up biochemical reactions in living organisms. As catalysts, enzymes are only required in very low concentrations, and they speed up reactions without themselves being consumed during the reaction.

Enzymes typically have common names which refer to the reaction that they catalyze, with the suffix “-ase” (e.g., oxidase, dehydrogenase, carboxylase), although individual proteolytic enzymes generally have the suffix “-in” (e.g., trypsin, chymotrypsin, papain). Often the common name also indicates the substrate on which the enzyme acts (e.g., glucose oxidase, alcohol dehydrogenase, pyruvate decarboxylase). However, some common names (e.g., invertase, diastase, catalase) provide little information about the substrate, the product, or the reaction involved.

Amino acid-based enzymes are globular proteins that range in size from less than 100 to more than 2000 amino acid residues. These amino acids can be arranged as one or more polypeptide chains that are folded and bent to form a specific three-dimensional structure, incorporating a small area known as the active site, where the substrate actually binds. The active site may involve only a small number (less than 10) of the constituent amino acids. The shape and charge properties of the active site enables enzyme binding to a single type of substrate molecule, so that the enzyme is able to demonstrate considerable specificity in its catalytic activity.

Enzymes do not alter the equilibrium (i.e., the thermodynamics) of a reaction. This is because enzymes do not fundamentally change the structure and energetics of the product(s) and reagent(s), but rather they simply allow the reaction equilibrium to be attained more rapidly.

As used herein and unless otherwise stated, amino acid residues of the enzyme within a first threshold, such as 7.0 Å, from the substrate when the substrate is bound to the enzyme (i.e., when the ES complex is formed as shown below) constitute the enzyme catalytic center, which is also referred to herein as active site region. As used herein and unless otherwise stated, amino acid residues of the enzyme located beyond a second threshold, such as 9.0 Å, from the substrate when the substrate is bound to the enzyme constitute the enzyme surface.

An enzyme-catalyzed reaction may proceed through three stages as follows:
E+S→ES complex→E+P
   The ES complex represents a position where the substrate (S) is bound to the enzyme (E) such that the reaction (whatever it might be) is made more favorable. As soon as the reaction has occurred, the product molecule (P) dissociates from the enzyme, which is then free to bind to another substrate molecule. At some point during this process the substrate is converted into an intermediate form (often called the transition state) and then into the product.

The exact mechanism whereby the enzyme acts to increase the rate of the reaction differs from one system to another. However, the general principle is that by binding of the substrate to the enzyme, the reaction involving the substrate is made more favorable by lowering the activation energy of the reaction by making it energetically easier for the transition state to form. In the presence of an enzyme catalyst, the formation of the transition state is energetically more favorable, thereby accelerating the rate at which the reaction will proceed, but not fundamentally changing the energy levels of either the reactant or the product.

As used herein, “efficiency” or “activity” or “catalytic activity” in the context of an enzyme refers to the ability of the enzyme to perform its catalytic function under given reaction conditions (such as temperature, pH, or ionic strength). Typically, such efficiency is indicated by the amount of converted substrate or generated product under given reaction conditions. In further embodiments, the remaining substrate or the generated product can be measured experimentally, such as using spectrophotometry if the substrate or the product reflecting or transmitting certain wavelength of light, fluorescence labeling and detection, or radiolabeling coupled with magnetic resonance spectroscopy (MRS) or magnetic resonance imaging (MRI), mass spectrometry. In yet further embodiments, the efficiency can be indicated by one or more of the constants as detailed herein, such as k_{cat} or K_{M} or k_{cat}/K_{M}.

The enormous catalytic activity of enzymes can perhaps best be expressed by a constant, k_{cat}, that is variously referred to as the turnover rate, turnover frequency, or turnover number. This constant represents the number of substrate molecules that can be converted to product by a single enzyme molecule per unit time (usually per minute or per second).

The Michaelis constant (K_{M}) is equal to the substrate concentration at which the enzyme converts substrates into products at half its maximal rate and hence is related to the affinity of the substrate for the enzyme.

The specificity constant (also known as kinetic efficiency) expressed by k_{cat}/K_{M} is a measure of how efficiently an enzyme converts substrates into products.

As used herein, “stability” in the context of an enzyme refers to the capacity of an enzyme to remain active following exposure to a particular set of conditions (e.g., temperature, pH, ionic concentration, inhibitory agents, etc.). For example, an enzyme mutant that exhibits enhanced stability relative to a wild-type enzyme, exhibits a smaller loss of activity upon exposure to a set of conditions than the wild-type enzyme.

All enzymes may be described by a four-part Enzyme Commission (EC) number, based on the chemical reactions they catalyze. If different enzymes (for instance from different organisms) catalyze the same reaction, then they receive the same EC number.

The first part of the EC number refers to the reaction that the enzyme catalyzes. There are seven categories of such reactions: oxidoreductases (which catalyze oxidation/reduction reactions), transferases (which catalyze transfer of a functional group from one substance to another), hydrolases (which catalyze formation of two products from a substrate by hydrolysis), lyases (which catalyze non-hydrolytic addition or removal of groups from a substrate), isomerases (which catalyze intramolecular rearrangement such as isomerization), ligases (which catalyze the joining of two molecules), and translocases (which catalyze the movement of ions or molecules across membranes or their separation within membranes).

The remaining digits of the EC number have different meanings according to the nature of the reaction identified by the first digit. For example, within the oxidoreductase category, the second digit denotes the hydrogen donor and the third digit denotes the hydrogen acceptor.

Non-limiting examples of oxidoreductases include oxidases, dehydrogenases, peroxidases, hydroxylases, oxygenases, and reductases.

Non-limiting examples of transferases include single carbon transferases; aldehyde and ketone transferases; acyl transferases; glycosyl, hexosyl, and pentosyl transferases; alkyl and aryl transferases; nitrogenous transferases; phosphorus transferases (e.g., phosphorylases or kinases); sulfur transferases; selenium transferases; and metal transferases.

Non-limiting examples of hydrolases include esterases, such as nucleases, phosphodiesterases, lipases, and phosphatases; DNA glycosylases; glycoside hydrolases; proteases/peptidases; and acid anhydride hydrolases, such as helicases and GTPases.

Non-limiting examples of lyases include decarboxylases, aldehyde lyases, oxo acid lyases, dehydratases, adenylyl cyclases, guanylyl cyclases, and ferrochelatases.

Non-limiting examples of isomerases include racemases, epimerases, cis-trans isomerases, intramolecular oxidoreductases, intramolecular transferases, and intramolecular lyases.

Non-limiting examples of ligases include ubiquitin ligases and chelatases.

Non-limiting examples of translocases include ATPases.

As used herein, “wild-type” or “WT” defines the cell, composition, tissue, or other biological material as it exists in nature.

As used herein, an “enzyme mutant” or a “mutant” refers to an enzyme obtained by substituting at least one amino acid residue with another amino acid residue or by deleting at least one amino acid residue or both in a wild-type enzyme. Accordingly, the amino acid residue substituted to or deleted is referred to herein as the “mutated amino acid residue” or “mutated residue” or “mutation” or “amino acid mutation”.

As used herein, an amino acid mutation is provided herein as two letters separated by an integer, such as A32S. The first letter provides the one letter code of the original amino acid residue to be mutated; while the last letter provides the mutation, such as A indicating a deletion, or one letter code of the mutated amino acid residue. In some embodiments, the integer is the numbering of the to-be-mutated amino acid residue in the amino acid sequence free of the mutation, optionally counting from the N terminus to the C terminus. In some embodiments, the integer is the numbering of the amino acid residue in a wild-type protein.

In some embodiments, the term “recombinant” refers to having at least one modification not normally found in a naturally occurring protein. In some embodiments, the term “recombinant” refers to being synthetized by human intervention. In further embodiments, an enzyme mutant as referred to herein is a recombinant protein.

A recombinant protein, such as an enzyme mutant, can be produced, expressing a polynucleotide encoding the protein in vivo or in vitro. For example, a recombinant protein can be produced by inserting a polynucleotide, encoding the protein into an appropriate expression vector, introducing the vector into an appropriate host cell, culturing the host cell under conditions for expressing the protein, and isolating the expressed protein. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; and Mayer and Walker eds. (1987) Immunochemical Methods in Cell and Molecular Biology (Academic Press, London).

As used herein, “homology” refers to sequence similarity between a reference sequence and at least a fragment of a second sequence. Homologs may be identified by any method known in the art, preferably, by using the BLAST tool to compare a reference sequence to a single second sequence or fragment of a sequence or to a database of sequences.

The terms “identical” or percent “identity,” in the context of two or more polypeptide sequences, refer to two or more sequences or subsequences that are the same. Two sequences are “substantially identical” if two sequences have a specified percentage of amino acid residues that are the same (i.e., 29% identity, optionally 30%, 40%, 45%, 50%, 55%, 60%, 65%, 70%, 75%, 80%, 85%, 90%, 95%, 99%, or 100% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 10 amino acids in length, or more preferably over a region that is 20, 50, 200, or more amino acids in length.

Examples of algorithms and methods of alignment of sequences for comparison include the algorithm of Myers and Miller, CABIOS 4:11 17 (1988); the local homology algorithm of Smith et al., Adv. Appl. Math. 2:482 (1981); the homology alignment algorithm of Needleman and Wunsch, J. Mol. Biol. 48:443 453 (1970); the search-for-similarity-method of Pearson and Lipman, Proc. Natl. Acad. Sci. 85:2444 2448 (1988); the algorithm Karlin and Altschul Proc. Natl. Acad. Sci. USA 90:5873 5877 (1993), although other methods may be utilized.

For sequence comparison, typically one sequence acts as a reference sequence, to which test sequences are compared, which may be done in a pairwise manner. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For BLASTP program, the default parameters are Gap opening penalty=11 and Gap extension penalty=1.

A “comparison window,” as used herein, includes reference to a segment of any one of the number of contiguous positions including, but not limited to, from 20 to 600, usually about 50 to about 200, more usually about 100 to about 150, in which a sequence may be compared to a reference sequence of the same number of contiguous positions after the two sequences are optimally aligned. In some implementations, optimal alignment of sequences for comparison can be conducted.

“Optimal alignment” refers to an alignment giving the highest percent identity score, for example, calculated using an algorithm or program as disclosed herein. For example, global protein alignments via the Needleman-Wunsch algorithm and local protein alignments via the Smith-Waterman algorithm use a substitution matrix to assign scores to amino-acid matches or mismatches, and a gap penalty for matching an amino acid in one sequence to a gap in the other. In standard dynamic programming of these alignments, the score of each amino acid position is independent of the identity of its neighbors, and therefore base stacking effects are not taken into account. Accordingly, optimal alignment of two sequences is the alignment that maximizes the sum of pair-scores less any penalty for gaps.

The optimal alignment, the homology, and the percent identity can be determined using software programs. Various parameters, including default parameters, may be used for alignment. One such alignment program is BLAST, using default parameters. In particular, preferred programs are BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

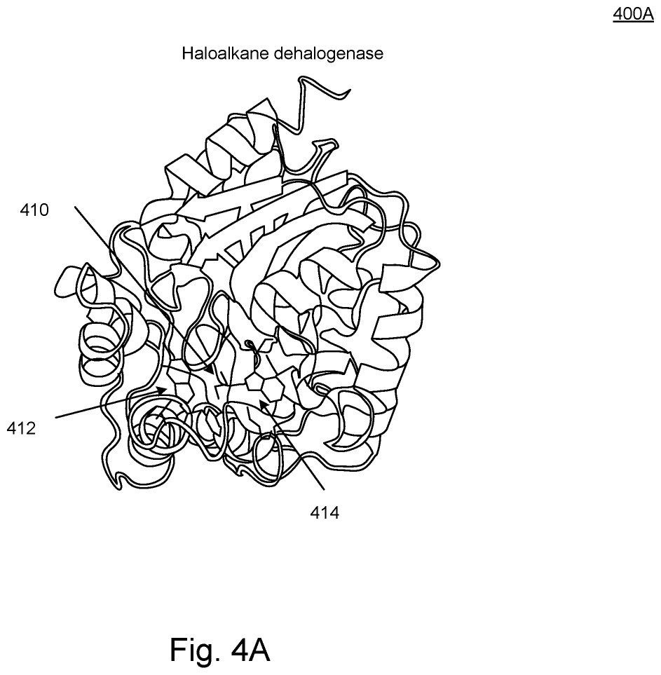

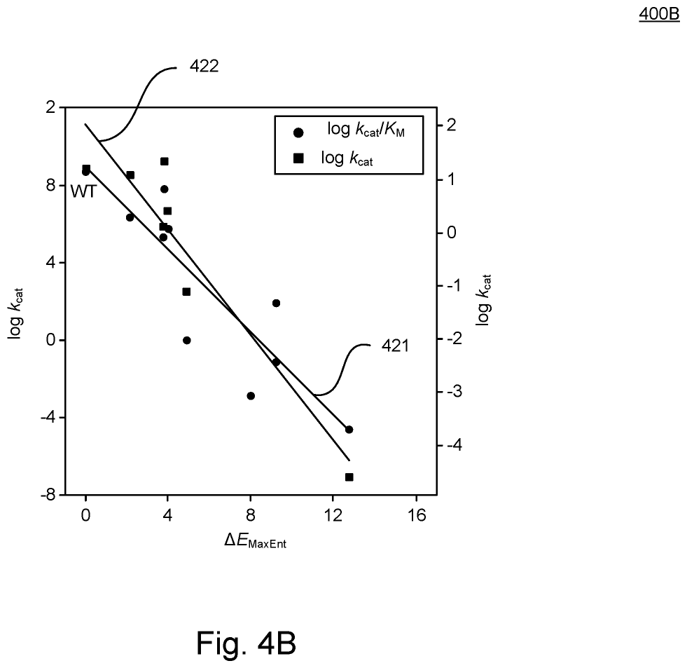

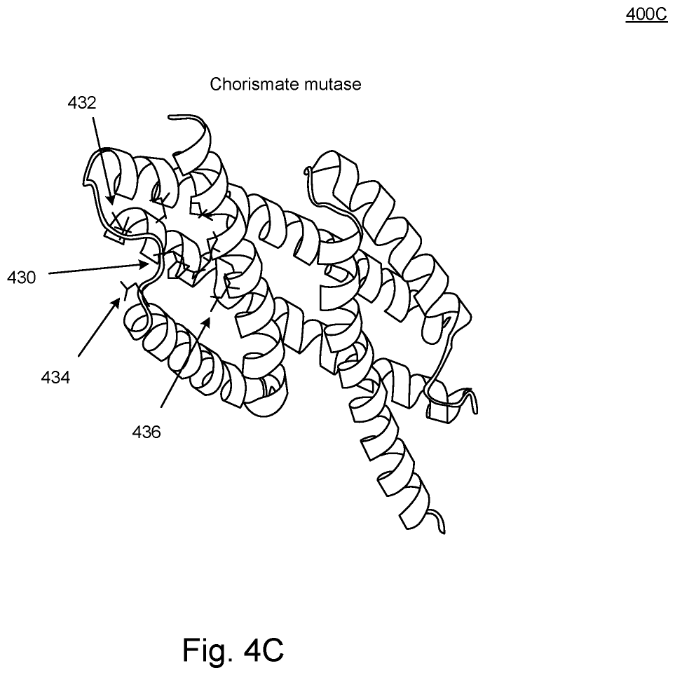

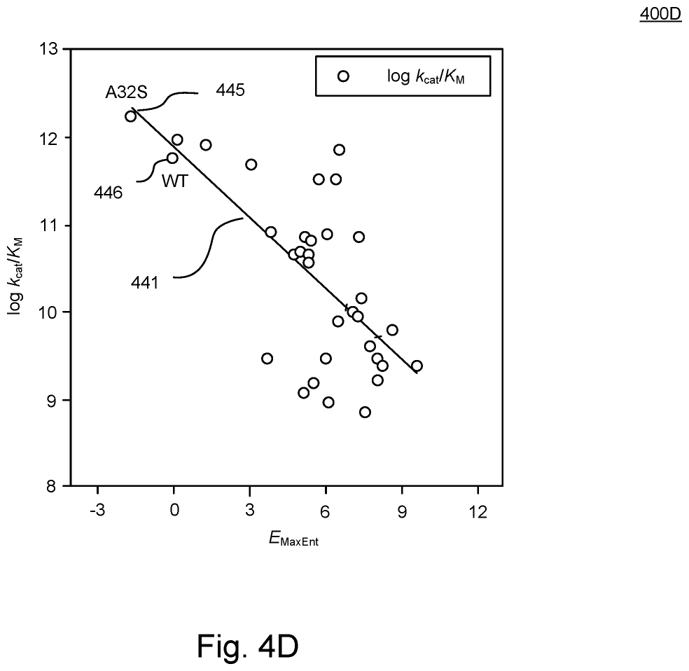

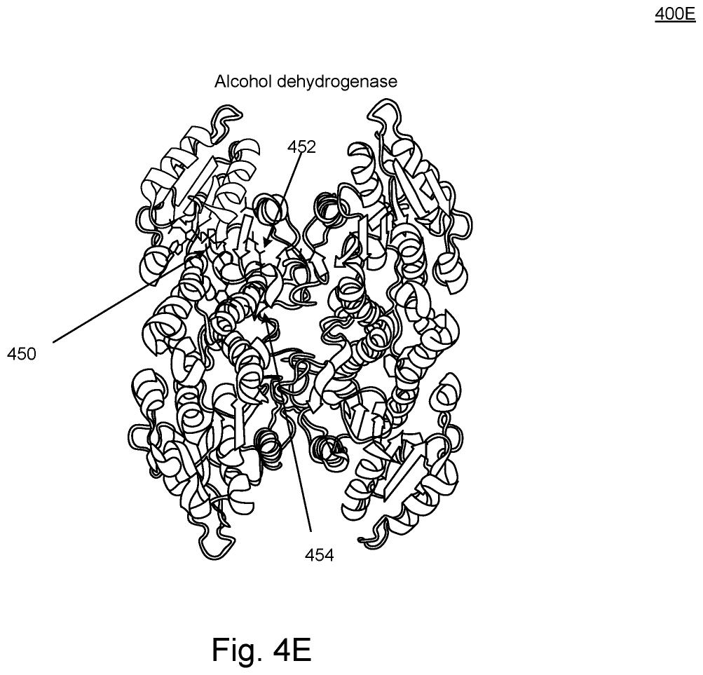

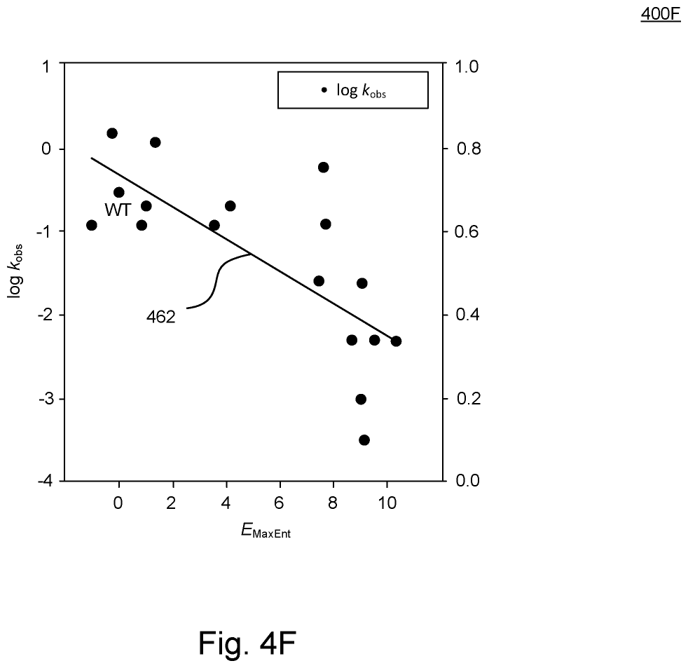

Embodiments of the present disclosure relate to systems and methods for generating new enzymes with either higher efficiency or higher stability versus wild-type enzymes. In some embodiments, a method for producing enzyme mutants includes generating a maximum entropy model based on homologous sequences of wild-type enzymes. Information on a plurality of enzyme mutants may be obtained. A mutant of an enzyme can arise when a mutation occurs at a residue. For example, A32S mutant is shown in FIG. 4D. That means, a mutation occurs such that the 32nd residue changed from alanine (abbreviated as A) to Serine (abbreviated as S). Therefore, A32S is a mutant and there is one residue difference from the WT (e.g., the hamming distance of sequences between A32S and WT is one). A distance between a mutated amino acid residue (which is also referred to herein as a mutation or a mutated residue or an amino acid mutation) of the plurality of enzyme mutants and a corresponding substrate when the substrate is bound to the mutant is compared to a first threshold.

In some embodiments, the first threshold is defined as a distance from the substrate within which the residue constitutes the enzyme catalytic center. If the distance is less than or equal to the first threshold, the mutant may be associated with or added to a first candidate set. Based on the maximum entropy model, a statistical energy of each mutant of the first candidate set of enzyme mutants is determined. Based on the determined statistical energies, a first set of enzyme mutants (with improved efficiency) among the first candidate set of enzyme mutants is identified. Similarly, a distance between a residue of each mutant of the plurality of enzyme mutants and a corresponding substrate is compared to a second threshold. In some embodiments, the second threshold is defined as a distance from the substrate beyond which the residue constitutes the enzyme catalytic surface. If the distance is greater than the second threshold, the mutant may be associated with or added to a second candidate set. Based on the maximum entropy model, a statistical energy of each mutant of the second candidate set of enzyme mutants is determined. Based on the determined statistical energies, a second set of enzyme mutants (with improved stability) among the second candidate set of enzyme mutants is identified.

In some embodiments, the distance between the mutated amino acid residue of the enzyme mutant and the substrate is determined based on a three-dimensional structure of the substrate-bound mutant. In some embodiments, the WT enzyme structure can be used to approximate the structure of an enzyme mutant. Such three-dimensional structure can be determined by various methods, such as X-ray, nuclear magnetic resonance (NMR) or electron microscopy (EM). In further embodiments, the three-dimensional structure is available from a Protein Data Bank (PDB) accessible at www.rcsb.org, www.ebi.ac.uk/pdbe, pdbj.org, or bmrb.io or other such database.

In other embodiments, the distance between (i) the mutated amino acid residue of the enzyme mutant and (ii) the substrate when the substrate is bound to the mutant is determined as the distance between (i) the amino acid residue of a wild-type enzyme corresponding to the mutated amino acid residue of the enzyme mutant and (ii) the substrate when the substrate is bound to the wild-type enzyme. In further embodiments, the distance between the amino acid residue of the wild-type enzyme and the substrate is determined based on a three-dimensional structure of the substrate-bound wild-type enzyme. Such three-dimensional structure can be determined by various methods, such as X-ray, NMR or EM. In yet further embodiments, the three-dimensional structure is available from a Protein Data Bank (PDB) as disclosed herein or other such database.

As used herein, a first amino acid residue in a first protein (such as an enzyme) “corresponding to” a second amino acid residue in a second protein refers to that the two residues are aligned with each other in a sequence alignment between the two proteins. Various programs are available for performing such sequence alignments, such as those disclosed above.

One problem relates to designing efficient and/or stable enzymes using computational modeling. Enzymes are extraordinary catalysts that play vital roles in nearly all biochemical processes. Designing efficient enzymes could help in solving catastrophic threats to humankind, including the energy crisis, environmental pollution, and food shortage. The use of computational modeling for enzyme design is very promising but is still not at the stage when it can guide sufficiently reliable enzyme design. Although computational enzyme design is of great importance, the advances utilizing physics-based approaches have been slow, and further progress is urgently needed. One promising direction is using machine learning, but strategies not utilizing the systems and methods discussed herein have been so far ineffective for predicting the catalytic power of enzymes. For example, many attempts have failed to focus on active regions or distinguish between active and inactive regions within the enzymes, and thus are unable to properly predict or grade efficacy of an enzyme.

Thus, it is crucial to exploit additional options for improving the design predictability. For example, a maximum entropy (MaxEnt) model taking epistasis into account can distill evolutionary information within a protein family, which are then correlated with residue-residue contact and fitness, partly leading to the breakthrough of de-novo protein structure prediction. For enzymes, a high correlation was found between the MaxEnt model and enzyme efficiency for beta-lactamase at the enzyme surface, but there is no such correlation for trypsin and dihydrofolate reductase. Similarly, prior attempts using such models, without the implementations discussed herein, were limited to only classifying a sequence as functional or not with the assistance of high-throughput experiments, but were unable to identify efficacy or stability. Using evolutionary information to design enzymes is still in its infancy, considering the complex interplay among various selection pressures applied to enzyme evolution. In particular, enzyme stability and activity may trade-off with each other. Accordingly, there is a need for additional machine learning options to design efficient and/or stable enzymes.

Moreover, naturally evolving enzymes can speed up chemical reactions by many orders of magnitude. Such great catalytic power reflects a very long evolutionary process that started at the emergence of life. There is a need to guide sufficiently reliable enzyme design based on naturally evolving enzymes.

To solve these problems, implementations of the systems and methods discussed herein provide statistical analysis of enzyme homologous sequences for enhancing computational enzyme design prediction. In some embodiments, an enzyme design system can infer a statistical energy from homologous sequences with maximum entropy (MaxEnt) principle such that the statistical energy significantly correlates with enzyme catalysis and stability at the active site region and more distant regions, respectively. These correlations can decode enzyme architecture and offer a connection between enzyme evolution and the physical chemistry of enzyme catalysis, and deepen the understanding of the stability-activity trade-off hypothesis for enzymes. In some embodiments, based on the strong correlations, the enzyme design system can provide a powerful way of guiding enzyme design. In particular, by focusing on active sites or functional regions within the enzyme, implementations of the systems and methods discussed herein may provide improved analysis and better predictions of enzyme stability and/or efficiency of catalytic functions.

According to certain aspects, implementations in the present disclosure relate to techniques for applying or using machine learning methods to provide an invaluable guide for enzyme designs. In some embodiments, an enzyme design system can use a MaxEnt model or principle which can provide a least-biased model for a sequence distribution by maximizing information entropy subject to statistics obtained from a multiple sequence alignment (MSA). The enzyme design system can design or identify enzymes with improved efficiency and/or stability using a MaxEnt model, based on the hypothesis that an enzyme catalytic center involved in a catalysis and transition state stabilization directly correlates with a selection pressure of enzyme efficiency in a way that can be captured by the MaxEnt model. In some embodiments, the enzyme design system can apply the MaxEnt model to show that (1) a significant correlation for a “catalytic center” between enzyme catalysis and statistical energy can be derived by applying the MaxEnt model; and that (2) the statistical energy correlates well with protein stability for remote regions (referred to here as “enzyme surface”), suggesting that a stable enzyme surface may be utilized for enzyme function. The enzyme design system can guide enzyme design using at least one of the correlations (1)-(2).

According to certain aspects, implementations in the present disclosure relate to techniques for generating or providing MSA data and training or learning a MaxEnt model based on the MSA data. In some embodiments, an enzyme design system can retrieve MSA data from a database in the form of extended strings of wild-type sequences, and then align different sequences, and/or homologous sequences from different species. In some embodiments, the enzyme design system can identify a pairwise frequency, e.g., frequency of pairwise correlations throughout sequence (any two different sites, not necessarily adjacent), and/or a single-order frequency for each amino acid. In some embodiments, the enzyme design system can generate an array of frequencies, which are then provided to a MaxEnt model. In some embodiments, the enzyme design system can use a Markov chain Monte Carlo method or simulation to sample sequence space. In some embodiments, the enzyme design system can use replica-exchange Markov chain Monte Carlo (MCMC) as an enhanced sampling method.

According to certain aspects, implementations in the present disclosure relate to techniques for outputting from a MaxEnt model, or determining based on the MaxEnt model, a score (e.g., energy value) for every given sequence of enzyme mutants, and selecting enzyme mutants with highest scoring (e.g., lowest E_{MaxEnt} value defined in Equation (1)). In some embodiments, an enzyme design system can determine, based on the MaxEnt model, a score for enzyme mutants at a catalytic center (e.g., those located within 7.0 Å from the substrate) to select enzyme mutants with improved efficiency. In some embodiments, the enzyme design system can determine, based on the MaxEnt model, a score for enzyme mutants at an enzyme surface (e.g., those located beyond 9.0 Å from the substrate) to select enzyme mutants with improved stability. In some embodiments, the enzyme design system can determine an energy value based on a model other than a MaxEnt model, e.g., other generative models.

According to certain aspects, implementations in the present disclosure relate to a method or a system for designing an enzyme mutant having a desirable efficiency or stability. In some embodiments, an enzyme design system can generate, determine, or obtain a maximum entropy (MaxEnt) model as illustrated in the equation below by inputting statistics data calculated from a natural MSA or homologous sequences of wild-type enzymes

$${E}_{MaxEnt} ( S ) = {\sum }_{i} {h}_{i} {S}_{i} + {\sum }_{i > j} {J}_{ij} {S}_{i} {S}_{j} , \qquad ( Expression 1 )$$

$$Z = {\sum }_{S} {e}^{- {E}_{MaxEnt} ( s )} .$$

where the model provides a Boltzmann distribution P(S)=^{-E}^{_{MaxEnt}}^{(s)}/Z for each homologous sequence S. E_{MaxEnt}(S) is a statistical energy with effective temperature as unity, Z is a partition function, hᵢ is site energy, Jᵢⱼ is a pair-wise coupling between amino acids at two different residue sites. In some embodiments, the enzyme design system can generate a natural MSA, and the statistics can be calculated from the natural MSA. For example, inputs to the MaxEnt model (Equation (1)) may be statistics data calculated from the natural MSA, i.e., <Sᵢ>ₑₓₚ, <SᵢSⱼ>ₑₓₚ.

In some embodiments, other generative models, e.g., autoregressive model Boltzmann machine (e.g. restricted Boltzmann machine, deep belief network), variational autoencoder, generative adversarial network, flow-based generative model, or energy based model can be used instead of the maximum entropy model.

In some embodiments, an enzyme design system can calculate a statistical energy (e.g., E_{MaxEnt}(S)) of each of a plurality of enzyme mutants with mutated amino acid residues in or at a catalytic center, rank or order the calculated energies, and identify, as mutants with improved efficiency, one or more enzyme mutants with their energies lower than an energy of wild-type enzymes. In other words, the enzyme design system can obtain a set of enzyme mutants with improved efficiency by decreasing E_{MaxEnt}(S) or increasing P_{MaxEnt}(S) with respect to mutated amino acid residues at the catalytic center.

In some embodiments, an enzyme design system can calculate a statistical energy (e.g., E_{MaxEnt}(S)) of each of a plurality of enzyme mutants with mutated amino acid residues on or at an enzyme surface, rank or order the calculated energies, and identify, as mutants with improved stability, one or more enzyme mutants with their energies lower than an energy of wild-type enzymes. In other words, the enzyme design system can obtain a set of enzyme mutants with improved stability by decreasing E_{MaxEnt}(S) or increasing P_{MaxEnt}(S) with respect to mutated amino acid residues at the enzyme surface.

In some embodiments, the enzyme design system can use generative models instead of a MaxEnt model. For example, instead of P(S) (or P_{MaxEnt}(S)) and E_{MaxEnt}(S), P_{Generative}(S) and E_{Generative}(S) can be obtained based on a particular generative model, and used to perform the above-noted procedure to obtain enzyme mutants with improved efficiency and/or enzyme mutants with improved stability.

According to certain aspects, implementations in the present disclosure relate to a method or a system for producing and/or using the enzyme obtained with the above-noted procedure. In some embodiments, a recombinant enzyme including at least one non-naturally occurring amino acid mutation can be produced and/or used. The recombinant enzyme has a statistical energy (e.g., E_{MaxEnt}(S); S is a homologous sequence of the recombinant enzyme) lower than a statistical energy of an enzyme that does not include the at least one non-naturally occurring amino acid mutation, where the statistical energy (E_{MaxEnt}(S)) is calculated following a maximum entropy model represented by the above-noted Equation (1) and generated by inputting homologous sequences of wild-type enzymes.

Embodiments in the present disclosure can have the following advantages. First, some embodiments can provide useful techniques for improving enzyme catalysis using the relationship between enzyme evolution and catalysis by correlating E_{MaxEnt} obtained from natural homologous sequences with the catalytic power of different enzymes. It is found that the correlation is significant for the catalytic center, and an enzyme design system according to some embodiments, can adopt the finding to guide enzyme design. As the catalytic center and enzyme surface face different selection pressures, the enzyme design system can improve enzyme catalysis by optimizing the catalytic center instead of the enzyme surface using evolutionary information.

Second, some embodiments can provide useful techniques for improving enzyme catalysis using the correlation between an energy (e.g., E_{MaxEnt}) obtained from natural homologous sequences and the melting temperature Tₘ. Alcohol dehydrogenase provides strong evidence for the enzyme activity-stability trade-off around substrate. For enzyme surface, E_{MaxEnt} and Tₘ are inversely correlated, while Tₘ is directly correlated with enzyme efficiency. This seems to contradict the idea that catalytic preorganization costs folding energy. However, the folding energy as expressed by Tₘ is related to the stability of the entire enzyme, and the preorganization can be determined by the folding of a limited part of the enzyme. Therefore, the enzyme design system can improve the stability of enzyme catalysis by reducing the energy (E_{MaxEnt}) on or at the enzyme surface. In some embodiments, for example, for cases where evolutionary information is not sufficient or for new catalytic reactions, an enzyme design system can improve the performance of the MaxEnt model by combining with empirical valence bond (EVB) calculations to model the catalytic power and double-screen the design to increase the success rate for enzyme design.

Third, some embodiments can provide useful techniques for extending the enzyme design by directed evolution. An enzyme design system according to some embodiments, can successfully decode enzyme architecture and connect enzyme evolution with enzyme catalysis. Such a connection can help to bridge evolutionary biology and enzymology. The high-throughput and predictability from the MaxEnt model, combined with experimental validation and computational modeling, can push enzyme studies to a systems level. The MaxEnt model can be combined with experimental directed evolution by suggesting a good starting point for the experiment. Moreover, embodiments of the present disclosure can be used to trace the moves in directed evolution by following the prediction of the MaxEnt model. In this manner, an enzyme design system can both improve enzyme efficiency and enzyme stability by focusing on the different parts of enzymes. Furthermore, the results here call attention to integrating domain knowledge in physical chemistry into machine learning models when studying biomolecules.

Fourth, some embodiments can provide useful techniques for commercially designing enzymes with predicted efficiency and/or stability without in vitro testing. Embodiments of the present application can be applied to many product families that have both a catalytic center and a surface element.

Before turning to the figures, which illustrate certain embodiments in detail, it should be understood that the present disclosure is not limited to the details or methodology set forth in the description or illustrated in the figures.

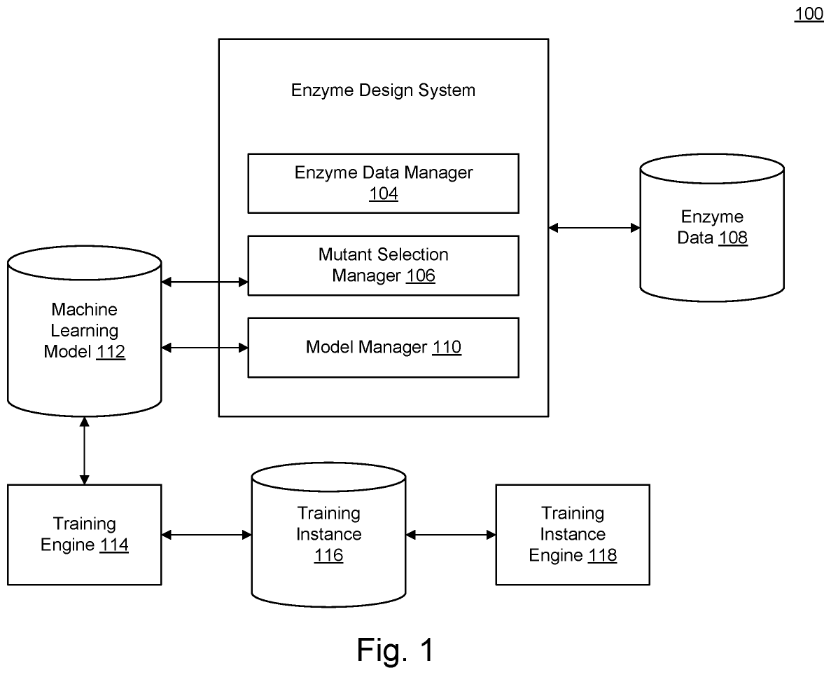

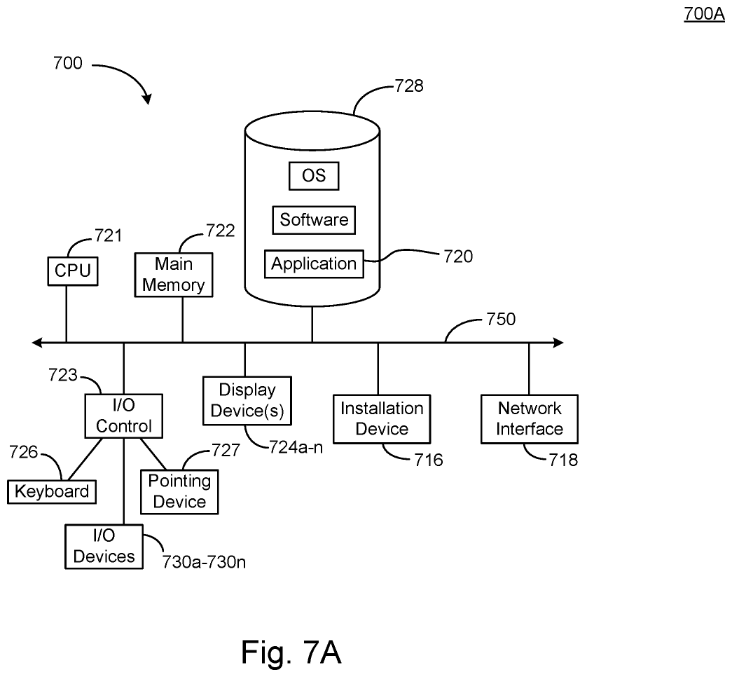

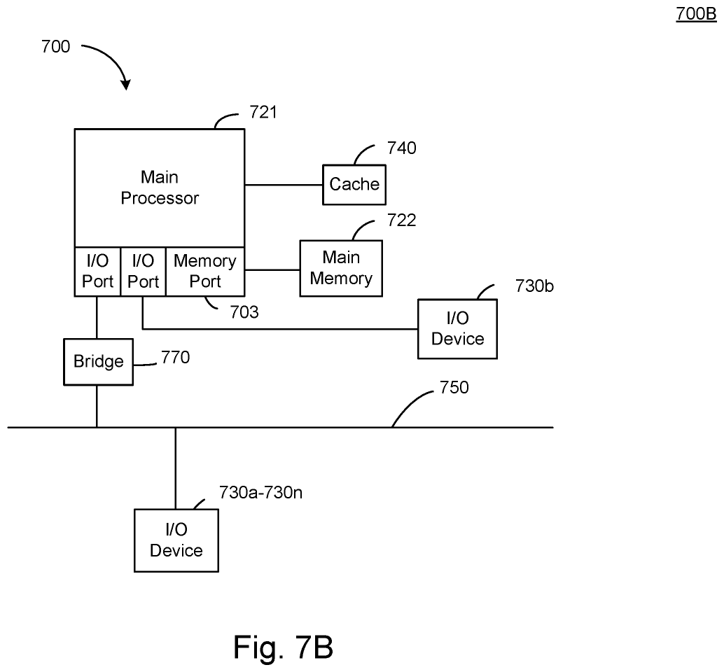

FIG. 1 is a block diagram illustrating an example of an enzyme design system 100 for training and/or using a machine learning model according to some implementations. The enzyme design system 100 can design or identify enzyme mutants with improved efficiency and/or stability using a machine learning model. In some implementations, enzyme design system 100 may include an enzyme data manager 104, a mutant selection manager 106, a model manager 110, a training engine 114, and a training instance engine 118. The enzyme data manager 104, mutant selection manager 106, model manager 110, training engine 114, and training instance engine 118 are example components in which techniques described herein may be implemented and/or with which systems, components, and techniques described herein may interface. The operations performed by one or more components 104, 106, 110, 114, and 118 of FIG. 1 may be distributed across multiple computing systems (e.g., multiple computing devices, each device having configuration similar to that of computing device 700 in FIG. 7A and FIG. 7B). In some implementations, one or more aspects of components 104, 106, 110, 114, and 118 may be implemented with software or hardware combined into a single system (e.g., computing device 700). For example, in some of those implementations, aspects of the enzyme data manager 104 may be combined with aspects of the mutant selection manager 106. Components in accordance with many implementations may each be implemented in one or more computing devices that communicate, for example, through a communication network. A communication network may include a wide area network such as the Internet, one or more local area networks (“LAN”s) such as Wi-Fi LANs, mesh networks, etc., and one or more bus subsystems. A communication network may optionally utilize one or more standard communication technologies, protocols, and/or inter-process communication techniques.

The enzyme design system 100 can perform a variety of processing on enzyme data 108. In some implementations, the enzyme data 108 includes data relating to at least one of MSA, design sequences, enzyme catalytic data, enzyme mutants, efficiency, stability, or experimental data. The enzyme data manager 104 can process or generate data relating to structure of enzymes (e.g., a physical distance between an amino acid residue, such as each mutated amino acid residue, of an enzyme mutant and a substrate when the substrate is bound to the enzyme mutant) or data relating to MSA by accessing the enzyme data 108.

The mutant selection manager 106 can obtain information on enzyme mutants, calculate or determine a value or score of each mutant (e.g., statistical energy of each mutant) based on a machine learning model (e.g., generative models including a MaxEnt model), and select or identify one or more mutants based on the value or score.

The enzyme data manager 104 can calculate a distance between residue(s) of a mutant and a substrate. For example, the enzyme data manager 104 can access, determine or obtain protein (enzyme) structure for wild-type (WT), from an online database (e.g., PDB) which stores the position of each residue and the substrate. Then, the enzyme data manager 104 can calculate the distance between each residue and the substrate. If the residue is within the first threshold (e.g., 7.0 Å), this residue can be regarded or determined as the active site (or catalytic center). If the residue is beyond the second threshold, this residue can be regarded or determined as the enzyme surface. In this manner, the enzyme data manager 104 can calculate a distance between a residue and a substrate solely based on WT structure without knowing the crystal structure for the designed mutant, and therefore the selection of residues is solely based on WT structure. For example, there is a mutant A32S shown in FIG. 4C and FIG. 4D. This mutation occurs at position 32 in the WT. The enzyme data manager 104 can calculate the distance between the residue at position 32 which is alanine (A) and the substrate. Assuming this distance is 3.0 Å which is smaller than the first threshold, the mutant selection manager 106 can include the residue at position 32 into a first candidate set for improved efficiency. Then, the mutant selection manager 106 can sample these active sites (or the enzyme surface) while fixing the other residues and obtain the corresponding mutants which are candidates of designed enzymes. Then, the mutant selection manager 106 can select a first set of mutants, from among the first candidate set, as designed enzymes.

The model manager 110 can train a machine learning model 112. The machine learning model 112, in accordance with some implementations, can include generative models, e.g., MaxEnt model, Gaussian mixture model (and other types of mixture model), hidden Markov model, Bayesian network (e.g. naïve bayes, autoregressive model), averaged one-dependence estimators, latent Dirichlet allocation, Boltzmann machine (e.g. restricted Boltzmann machine, deep belief network), variational autoencoder, generative adversarial network, flow-based generative model, and energy based model. Additionally or alternatively, the machine learning model 112 can represent a variety of neural network models including feed forward neural networks, convolutional neural networks, recurrent neural networks, radial basis functions, other neural network models, as well as combinations of several neural networks. Training the machine learning model 112 in accordance with some implementations described herein can utilize the model manager 110, training engine 114, and training instance engine 118. Machine learning model 112 can be trained or learned for a variety of enzyme design tasks including selecting or identifying enzyme mutants with improved efficiency and/or stability, or predicting the catalytic power of enzymes, or guiding enzyme designs using one or more trained models.

The training instance engine 118 can generate training instances 116 (e.g., based on enzyme data 108) to train a neural network model. A training instance can include, for example, MSA data and/or protein sequence data. The training engine 114 may apply a training instance as input to machine learning model 112. In some implementations, the machine learning model 112 can be trained using at least one of supervised learning, unsupervised learning, or semi-supervised learning. In some embodiments, the training engine 114 may apply steepest gradient descent (SGD) for optimizing an objective function or parameters of the MaxEnt model, apply Markov chain Monte Carlo (MCMC) for sampling from a probability distribution, and/or apply message passing Interface (MPI) for utilizing parallel processing. Additionally or alternatively, the training engine 114 can compare the predicted model output with a known output from the training instance and, using the comparison, update one or more weights or parameters in the machine learning model 112.

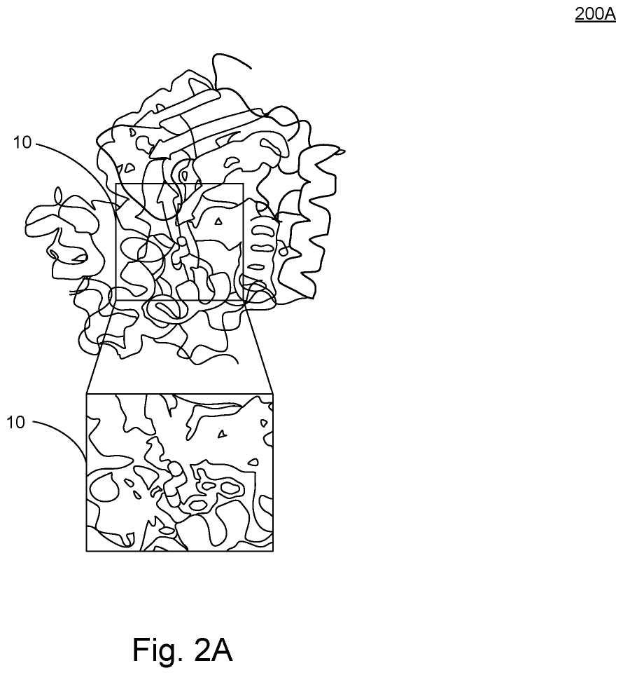

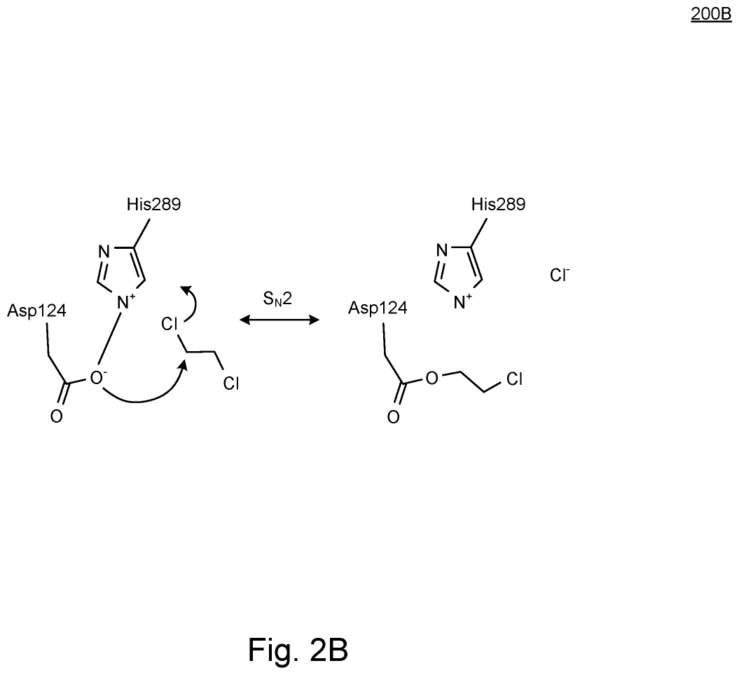

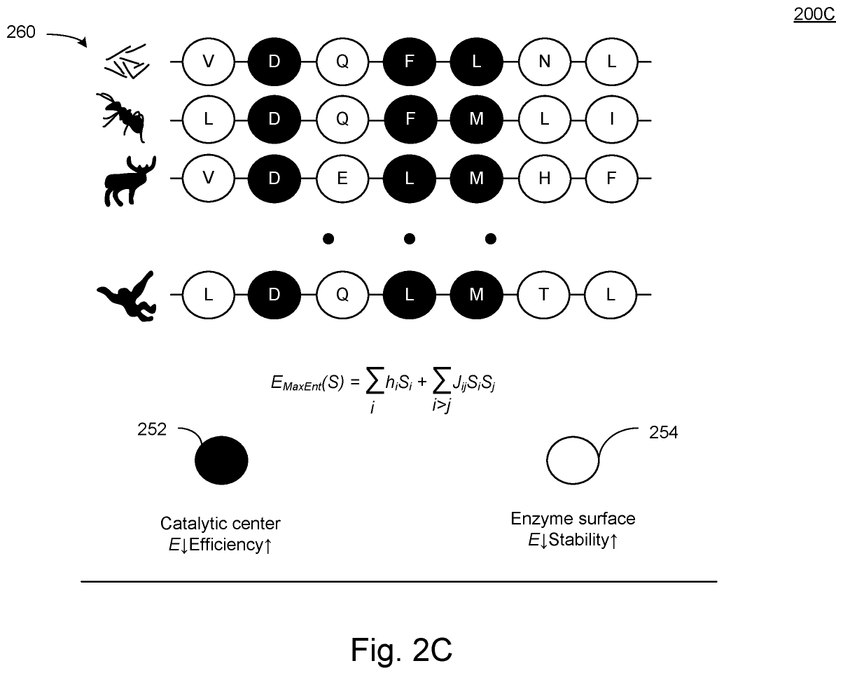

FIG. 2A to FIG. 2C are diagrams depicting an example of a maximum entropy model according to some implementations. An enzyme accelerates a chemical reaction by lowering the activation energy using mainly the residues in a catalytic center. For example, when Haloalkane dehalogenase (PDB) 200A (e.g., protein data bank: 2dhc) is used as an example to illustrate enzyme catalysis and reaction mechanism, the residues in a catalytic center 10 (e.g., within a distance of 7.0 Å from the substrate) are highlighted (see FIG. 2A); and the scheme of an S_{N}2 step 200B is illustrated using the substrate of 1,2-dichloroethane (see FIG. 2B).

In some embodiments, a maximum entropy (MaxEnt) model 200C for enzyme sequences can connect enzyme evolution and function. For example, the MaxEnt model 200C can connect enzyme evolution to the physical chemistry of enzyme catalysis. In some embodiments, an enzyme design system (e.g., system 100 in FIG. 1) can learn a pair-wise MaxEnt model from an MSA, and associate each protein sequence (S) with statistical energy (E_{MaxEnt}) following the Boltzmann distribution, as shown in the Equation (1). The enzyme design system can use the finding that decreasing the statistical energy significantly correlates with increasing enzyme efficiency and stability in the catalytic center 252 and enzyme surface 254, respectively (see FIG. 2C).

It is also found that homologous enzyme sequences from different species 260 share the same evolutionary origin; and that the natural sequence variation within an enzyme family is constrained by different factors, including its physical chemistry. Therefore, distilling evolutionary information from MSA of an enzyme family could shed light on enzyme 3D structure and function. In some embodiments, due to limited homologous sequences and high computational cost, the MaxEnt model may be truncated to consider pair-wise epistatic effect.

In some embodiments, an enzyme design system can use the MaxEnt model or principle to learn a least-biased model for the sequence distribution within a protein family P(S). Due to the computational cost and data availability, the model may be truncated to second order. Specifically, the information entropy of protein sequence H(S) may be maximized while constrained by the frequency of different amino acids at any site i (<Sᵢ>ₑₓₚ) and pairwise correlations between amino acids at any two different sites i,j (<Sᵢⱼ>ₑₓₚ) calculated from the MSA:

$$maximize H ( S ) = - {\sum }_{S} P ( S ) log P ( S ) \qquad ( Equation 2 )$$

$$subject to {〈 {S}_{i} 〉}_{e x p} = {\sum }_{S} P ( S ) {S}_{i} ,$$

$${〈 {S}_{i} {S}_{j} 〉}_{e x p} = {\sum }_{S} P ( S ) {S}_{i} {S}_{j} , for all i , j .$$

The MaxEnt model may be provided in the above-noted Equation 1. In some embodiments, the E_{MaxEnt} may be shifted by a constant so that the wild-type (WT) has a zero value and will not affect any results due to gauge invariance. A lower E_{MaxEnt} for a sequence indicates a higher probability to appear during evolution and might reflect a particular evolutionary advantage. The statistical energy E_{MaxEnt} is also a spin-glass Hamiltonian which has enormous local frustrations. The parameterization, which may require rigorous sampling of the model, may be thus highly non-trivial, especially for large proteins with hundreds of residues. In some implementations, the pseudo-likelihood (PLL) approximation can be used to quickly estimate the parameters. Embodiments of the present disclosure can develop and/or use an efficient code that marries different computational advancements to sample the Hamiltonian rigorously.

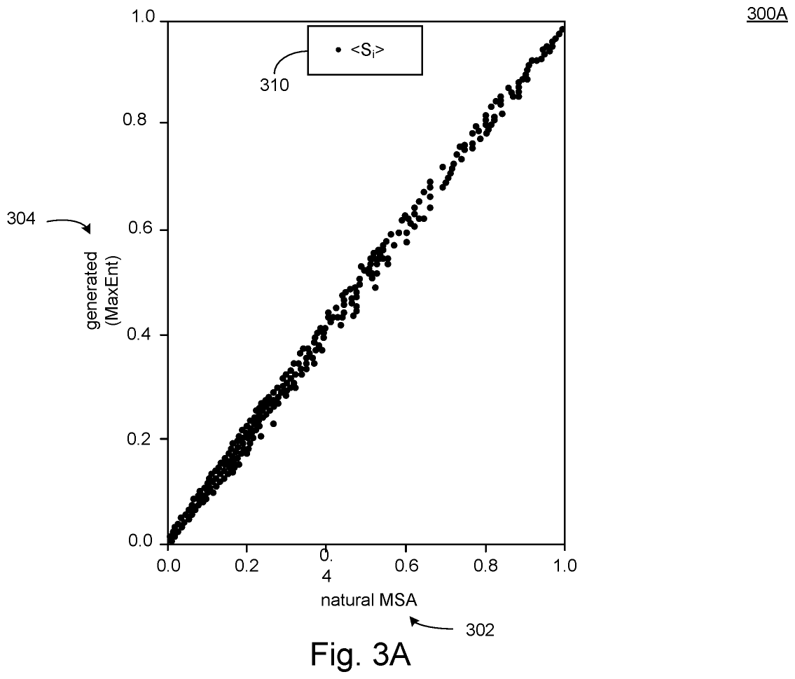

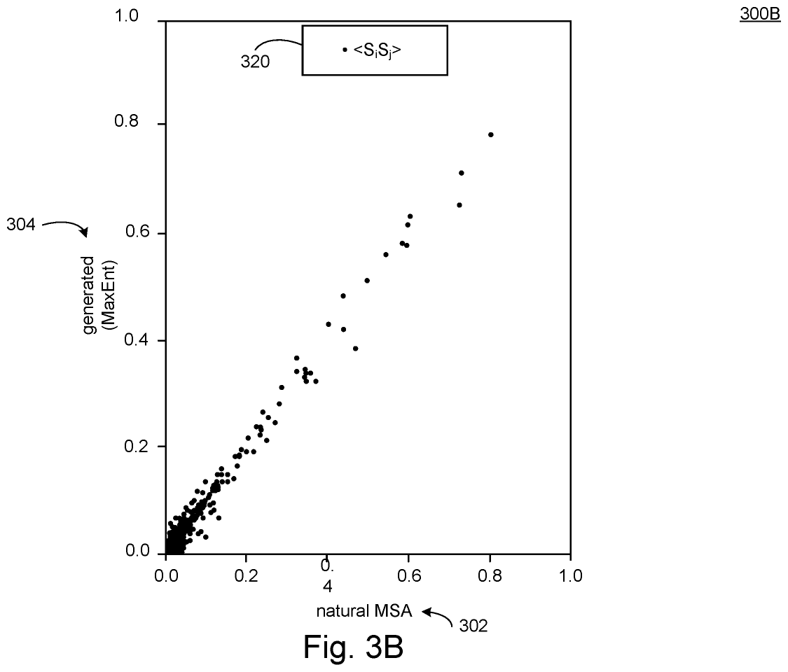

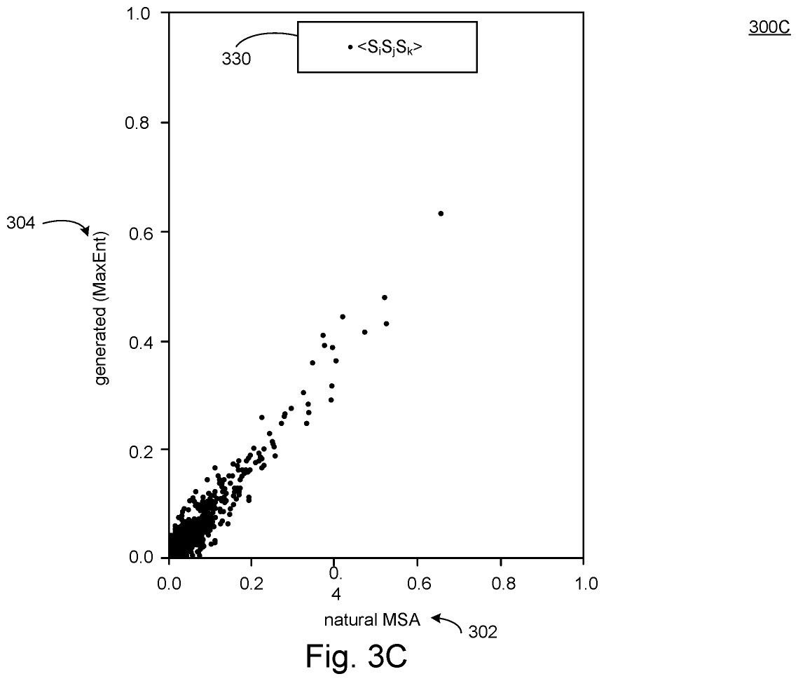

FIG. 3A to FIG. 3C are graphs 300A, 300B, and 300C, respectively, showing comparison of sequence statistics obtained from a maximum entropy model and natural multiple sequence alignment (MSA), according to some implementations. FIG. 3A to FIG. 3C show that reproduction and prediction of natural MSA statistics validate the MaxEnt model according to some embodiments, of generated MaxEnt 304 as a function of natural MSA 302. FIG. 3A, FIG. 3B, and FIG. 3C show comparison results with respect to <Sᵢ> 310, <SᵢSⱼ> 320 and <SᵢSⱼSₖ> 330, respectively. The <Sᵢ> 310 and <SᵢSⱼ> 320 are explicitly used as constraints in the parameterization while <SᵢSⱼSₖ> 330 is not. To avoid overplotting, only 1.5 million randomly sampled data are shown for both <SᵢSⱼ> 320 and <SᵢSⱼSₖ> 330. The reproduction of <Sᵢ> 310 and <SᵢSⱼ> 320, and prediction of <SᵢSⱼSₖ> 330 validates the MaxEnt model according to some embodiments.

The MaxEnt model for protein sequence is also named direct coupling analysis, evolutionary coupling (DCA), GREMLIN, and Boltzmann machine direct coupling analysis (bmDCA) elsewhere. Referring to Equation (1), the parameters Jᵢⱼ may quantify the direct coupling between residues at different sites, which are distinct from the pairwise correlations <Sᵢⱼ>ₑₓₚ. In this manner, an enzyme design system can use MaxEnt models to successfully perform protein structure prediction and quantify protein fitness upon mutation.

In some embodiments, an enzyme design system (e.g., system 100, model manager 110, or training engine 114 in FIG. 1) may learn parameters in the MaxEnt model (see Equation (1)) by minimizing the cross-entropy L(θ) between the sequence distribution in the MSA and the model distribution:

$$minimize L ( θ ) = {\sum }_{p} {F}_{p}^{e x p} {θ}_{p} - log Z ( θ ) , \qquad ( Equation 3 )$$

$$where {F}_{p}^{e x p} = \{ {〈 {S}_{i} 〉}_{e x p} , {〈 {S}_{i} {S}_{j} 〉}_{e x p} \} , θ = \{ {h}_{i} , {J}_{ij} \} .$$

In some embodiments, the derivative of the cross-entropy with respect to the parameters can be derived as:

$$\frac{\partial L}{\partial {θ}_{p}} = {F}_{p}^{exp} - \frac{\partial log Z ( θ )}{\partial {θ}_{p}} = {F}_{p}^{e x p} - {F}_{p}^{m o d e l} . \qquad ( Equation 4 )$$

In some embodiments, the parameters can be iteratively optimized using the steepest gradient descent (SGD) optimization with the following expression:

$${θ}_{p}^{t + 1} = {θ}_{p}^{t} - {α ( \frac{\partial L}{\partial {θ}_{p}} )}_{{θ}^{t}} . \qquad ( Equation 5 )$$

Now, embodiments of parameterization of the MaxEnt model will be described in more detail. The parameterization problem for the MaxEnt model is convex, and there are not non-global optima in the parameter space in principle. However, the optimization process is highly non-trivial in practice. The MaxEnt model may be a spin-glass model with enormous local frustrations. Thus, rigorously sampling from the model may require overcoming enormous energy barriers. In some embodiments, the MaxEnt model can adopt approximations, including mean-field approximation and/or pseudo-likelihood approximation. Although such approximations have already shown the impressive power of the MaxEnt model, the statistics cannot be well reproduced, indicating the sequence distribution is biased with these approximations. In some embodiments, more rigorous inference scheme can be used to overcome this drawback.

In some embodiments, an enzyme design system (e.g., system 100, model manager 110, or training engine 114 in FIG. 1) may implement or use a rigorous and efficient code for the parameterization which applies the advancements of different computational techniques, for example, replica exchange Markov chain Monte Carlo (MCMC), momentum-assisted steepest gradient descent (SGD), and Message Passing Interface (MPI) for parallel computing. The code can be used to parameterize the MaxEnt Hamiltonian from scratch or fine-tune the parameters obtained from approximation methods. The principles and details of the code are explained below.

(i) Replica exchange MCMC may be used as an enhanced sampling method. The enhancement can be achieved by constructing a series of replicas at different temperatures and swapping between different replicas based on Boltzmann distribution. The high-temperature replicas can accelerate the escaping from the local minima of the MaxEnt Hamiltonian. In a typical replica-exchange MCMC, there may be N replicas each in canonical ensemble associated with a different temperature Tᵢ. To preserve the canonical distribution, the swapping between any two replicas i,j may follow the Metropolis acceptance ratio:

$${P}_{a c c e p t :} = min \{ 1 , e ( \frac{1}{{τ}_{i}} - \frac{1}{{τ}_{j}} ) ( {E}_{i} ( S ) - {E}_{j} ( S ) ) \} . \qquad ( Equation 6 )$$

(ii) To further accelerate the parameterization and ensure the SGD is in the right direction during optimization, the enzyme design system can add or implement a momentum step when updating the parameters:

$${θ}_{p}^{t + 1} = {ϕ}_{p}^{t} - {α ( \frac{\partial L ( θ )}{\partial {θ}_{p}} )}_{{θ}^{t}} , \qquad ( Equation 7 )$$

$${ϕ}_{p}^{t + 1} = {θ}_{p}^{t} + β ( {θ}_{p}^{t + 1} - {θ}_{p}^{t} ) . \qquad ( Equation 8 )$$

(iii) Besides, the enzyme design system can include an MPI implementation, which enables a user to use multiple processors to converge the calculation of ensemble average faster.

In some embodiments, among a plurality of the protein families, the enzyme design system may use some number of replicas (e.g., 13 replicas) with temperatures evenly distributed between 0.8 and 2.0 in replica exchange MCMC. Each replica may last for 10⁵ steps before swapping. In some embodiments, sixteen processors may be used in the MPI implementation. In some embodiments, the learning rates α, β may be set such that α=0.001 and β=0.001. The enzyme design system may also use L₂-regularization to overcome the sampling noise from the MSA. In some embodiments, the weight decay factor may be 0.01 for all parameters.

Now, embodiments of application of the maximum entropy model to analysis of protein structure will be described in more detail.

A critical obstacle to examining the enzyme evolution-catalysis relationship may be the lack of enzyme catalytic data covering sufficient mutants. Directly measuring the catalytic parameters (k_{cat}, k_{cat}/K_{M} or k_{obs}) requires laborious biochemical assays. The experimental data (e.g., experimental data as enzyme data 108 in FIG. 1) may be thus relatively sparse. In some embodiments, data (e.g., enzyme data 108 in FIG. 1) or a database (e.g., a database including enzyme data 108) for enzyme efficiency upon mutation from published literature (Table S1-S9 below) may be curated, organized, or prepared so that an enzyme design system (e.g., system 100, model manager 110, or training engine 114 in FIG. 1) can process many amino acid mutations either in a catalytic center (defined as a location within 7.0 Å from the substrate, for example) or at an enzyme surface (defined as a location beyond 9.0 Å from the substrate, for example). The database may contain twelve enzyme-substrate pairs and/or protein stability data whenever available. For each pair, at least seven mutations measured in similar conditions (pH and temperature) may be collected. In some embodiments, the database includes many higher-order mutations (up to the tenth order). Meanwhile, it is confirmed that each enzyme has enough homologous sequences in the MSA to get statistically meaningful evolutionary information (Table S10 below). The enzymes used by the enzyme design system can cover various types of reactions identified by their different Enzyme Commission class number (Table S10 below).

FIG. 4A to FIG. 4F are diagrams depicting an example of correlation between a statistical energy and enzyme efficiency according to some implementations.

FIG. 4A to FIG. 4F show that the statistical energy obtained or calculated based on the MaxEnt model according to some embodiments correlates with enzyme efficiency at the catalytic center. FIG. 4A shows an example haloalkane dehalogenase 400A in which mutated residues 412, 414 and substrate(s) 410 are shown, and FIG. 4B shows its correlation results 400B (e.g., correlation between the statistical energy based on the MaxEnt model and enzyme efficiency such as the observed enzyme catalytic power in or at the catalytic center of the haloalkane dehalogenase). FIG. 4C shows an example chorismate mutase 400C in which mutated residues 432, 434, 436 and substrate(s) 430 are shown and only one unit of the dimeric chorismate mutase is highlighted, and FIG. 4D shows its correlation results 400D (e.g., correlation between the statistical energy based on the MaxEnt model and enzyme efficiency such as the observed enzyme catalytic power in or at the catalytic center of the chorismate mutase). FIG. 4E shows an example alcohol dehydrogenase 400E in which mutated residues 452, 454 and the cofactor NADP⁺ and catalytic triad 450 are shown and only one unit of the tetrameric alcohol dehydrogenase is highlighted, and FIG. 4F shows its correlation results 400F (e.g., correlation between the statistical energy based on the MaxEnt model and enzyme efficiency such as the observed enzyme catalytic power in or at the catalytic center of the alcohol dehydrogenase). For the alcohol dehydrogenase, the cofactor NADP⁺ and catalytic triad are shown in FIG. 4E because of the absence of substrate. PDB IDs used in rendering the structures are 2dhc (FIG. 4A); lecm (FIG. 4C); and 6tq5 (FIG. 4E).

FIG. 4A and FIG. 4B show correlation results of the haloalkane dehalogenase from Xanthobacter autotrophicus that catalyzes the conversion of toxic haloalkanes to alcohols. The correlation between the statistical energy E_{MaxEnt} and the observed enzyme catalytic power (expressed by both log k_{cat}/K_{M} (line 421) and log k_{cat} (line 422)) are evaluated. All the mutations (e.g., 412, 414 out of six mutations) are located at residues in the catalytic center with a mean distance of 3.4 Å from the substrate (e.g., 1,2-dichloroethane 410). Except for one double mutation, the other six are single mutations. The enzymatic rates span more than six orders of magnitude, posing great challenges for prediction methods. Nevertheless, as seen from FIG. 4B, the E_{MaxEnt} shows impressive correlations with log k_{cat}/K_{M} and log k_{cat} with values of −0.87 and −0.95, respectively.

FIG. 4C and FIG. 4D show correlation results of the catalytic center of chorismate mutase, which is widely used in enzyme mechanism and design studies. This enzyme transforms chorismite to prephenate in the pathway to produce tyrosine and phenylalanine, essential for plants, fungi, and bacteria. The enzyme mutations (e.g., 432, 434, 436) from Escherichia coli are 3.7 Å from the substrate on average. Here again, the correlations are significant, and the E_{MaxEnt} has a correlation value of −0.68 with log k_{cat}/K_{M} (line 441). The A32S mutation (445) stands out as the only mutant with increased efficiency relative to the WT (446), which is considered to be a unique experimental result.

For both haloalkane dehalogenase (FIG. 4A) and chorismite mutase (FIG. 4C), the mutants are mainly single mutations, while mutants in alcohol dehydrogenase (FIG. 4E) are higher-order mutations. FIG. 4E and FIG. 4F show correlation results of alcohol dehydrogenase from Starmerella magnolia in which nine of the 20 designs having experimental kinetic data are higher-order mutations up to the tenth order. The average distance between the mutations (e.g., mutated residues) and substrate is 6.2 Å (see FIG. 4E). The E_{MaxEnt}−log k_{obs} correlation (line 462) is −0.74 (see FIG. 4F) which shows that embodiments of the present disclosure can successfully predict such higher-order mutations. Interestingly, it is also found that the E_{MaxEnt} nearly perfectly correlates with melting temperature Tₘ (correlation value of 0.91) but with the opposite trend as the catalytic efficiency, supporting the activity-stability trade-off proposal. Also, the independent model without epistasis shows opposing trends as the MaxEnt model for alcohol dehydrogenase (see Table S12 below), arguing for the importance of epistasis in higher-order mutations.

Now, the generality of the above-noted findings (from FIG. 4A to FIG. 4F) is examined by considering an extensive set of enzymes summarized in Table 1. For all the mutations in the catalytic center, a strong correlation between the MaxEnt model and the catalytic effect is observed. The correlation appears to be insensitive to substrates for ketosteroid isomerase. For dihydrofolate reductase (DHFR), the strong correlation disappears when moving from the catalytic center to the enzyme surface. Such results confirm the hypothesis that the catalytic center evolved under the selection pressure of optimizing enzyme catalysis.

In addition, the correlation obtained here using the rigorous sampling is slightly stronger than those using PLL approximation; but both of them are better than the independent model (see Table S12-S13 below). The results obtained from the PLL approximation again confirm the above-noted findings.

TABLE 1
Correlation values between the maximum entropy model and enzyme efficiency/Tₘ

| | E_{MaxEnt} − | E_{MaxEnt} − | E_{MaxEnt} − | Distance to | Mutation | |
|---|---|---|---|---|---|---|
| Enzyme | log k_{cat}/K_{M} | log k_{cat} | Tₘ | substrate(Å) | region | Substrate |
| Haloalkane | −0.87 | −0.95 | | 3.4 ± 0.4 | catalytic | 1,2-dichloroethane |
| dehalogenase | | | | | center | |
| Chorismate | −0.68 | −0.51 | −0.20 | 3.7 ± 0.8 | catalytic | Chorismate |
| mutase | | | | | center | |
| Alcohol | −0.74 | | 0.91 | 6.2 ± 3.8 | catalytic | Cyclohexanol |
| dehydrogenase^{(a)} | | | | | center | |
| Triosephosphate | −0.62 | −0.69 | | 3.9 ± 1.6 | catalytic | DHAP |
| isomerase | | | | | center | |
| Ketosteroid | −0.74 | −0.75 | | 2.1 ± 0.5 | catalytic | 5(10)-estrene-3,17- |
| isomerase | | | | | center | dione |
| | −0.81 | −0.77 | | 3.4 ± 1.3 | catalytic | 5-androstenedione |
| | | | | | center | |
| Dihydrofolate | | −0.74 | | 5.4 ± 2.3 | catalytic | NADPH |
| reductase^{(b)} | | | | | center | |
| | | 0.08 | | 11.4 ± 3.1 | enzyme | |
| | | | | | surface | |
| Dihydrofolate | −0.42 | 0.16 | −0.65 | 10.6 ± 3.8 | enzyme | NADPH |
| reductase^{(c)} | | | | | surface | |
| Beta-lactamase | −0.57 | −0.48 | −0.68 | 10.4 ± 5.8 | enzyme | 6- |
| | | | | | surface | furylacrylpenicillanic |
| | | | | | | acid |
| | −0.37 | 0.04 | | 16.4 ± 4.8 | | ampicillin |
| Trypsin | 0.06 | −0.03 | −0.65 | 10.1 ± 5.2 | enzyme | Suc-AAPK-pNA |
| | | | | | surface | |

It is noted that (a) the enzyme kinetics is measured as log k_{obs} (Table S3 below) and collected from a reference; (b) data collected from a reference (Table S6 below); and (c) data collected from a reference (Table S7 below).

For the enzyme regions, which are at least 9.0 Å away from the substrate (referred to as “enzyme surface”), the correlation between E_{MaXEnt} and enzyme efficiency is not that much stronger or systematic, although in general there seems to be a negative correlation. Using beta-lactamase as an example (and discarding the substrate difference in two enzyme-substrate pairs), E_{MaxEnt} has a stronger correlation with enzyme catalysis for mutations closer to the substrate. This is consistent with the above-noted finding on the catalytic center. The rationale is that the enzyme surface region is not directly responsible for the evolution pressure of enzyme catalysis.

To better understand the physical nature of E_{MaXEnt}, the correlation between E_{MaXEnt} and the observed Tₘ (which is inversely related to the folding energy) is considered in Table 1. As seen from Table 1, there is a systematically negative correlation between E_{MaXEnt} and Tₘ for the enzyme surface, indicating that the MaxEnt model does reflect the protein stability for regions far away from where catalysis happens. It is reasonable since the MaxEnt model reflects the contact probability which can be considered as a generalized free energy function for protein folding.

It appears that the catalytic center and enzyme surface face different selection pressures. The statistical energy inferred from MSA strongly inversely correlated with enzyme efficiency and enzyme stability in the catalytic center and enzyme surface, respectively. The finding that a more stable enzyme surface could promote enzyme catalysis could also rationalize the growing evidence that it is possible to engineer remote mutations to improve catalysis (Table 1).

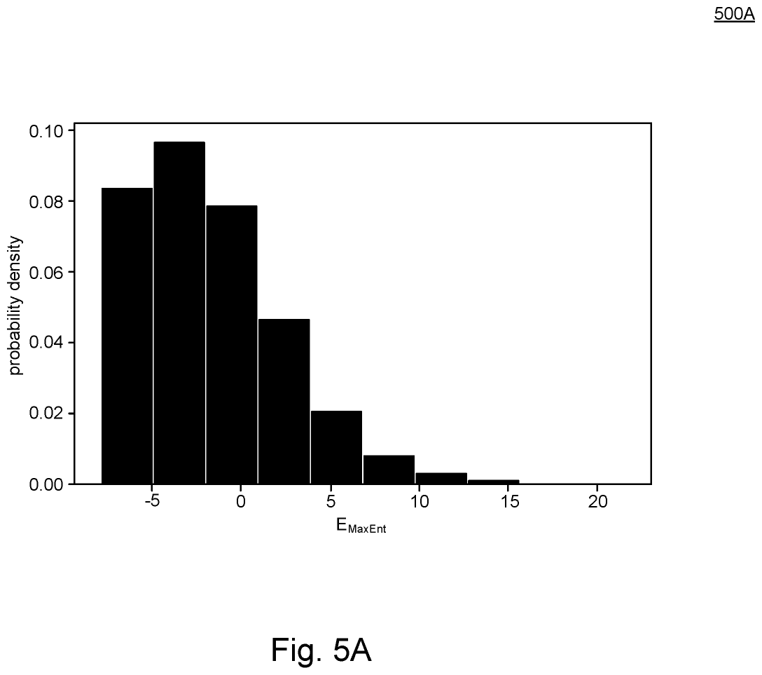

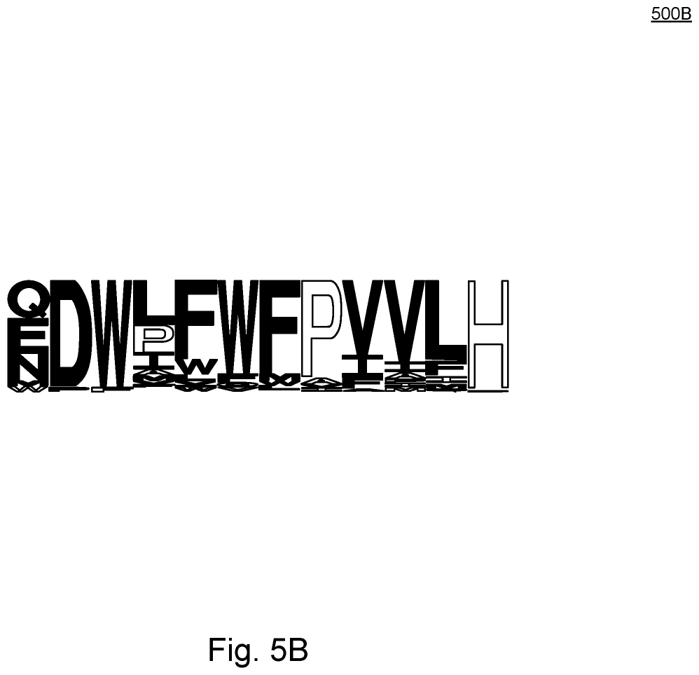

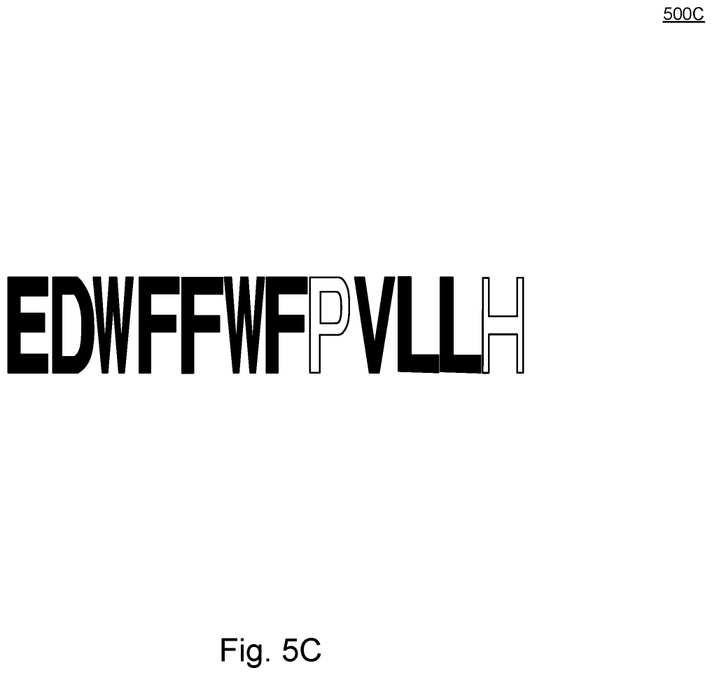

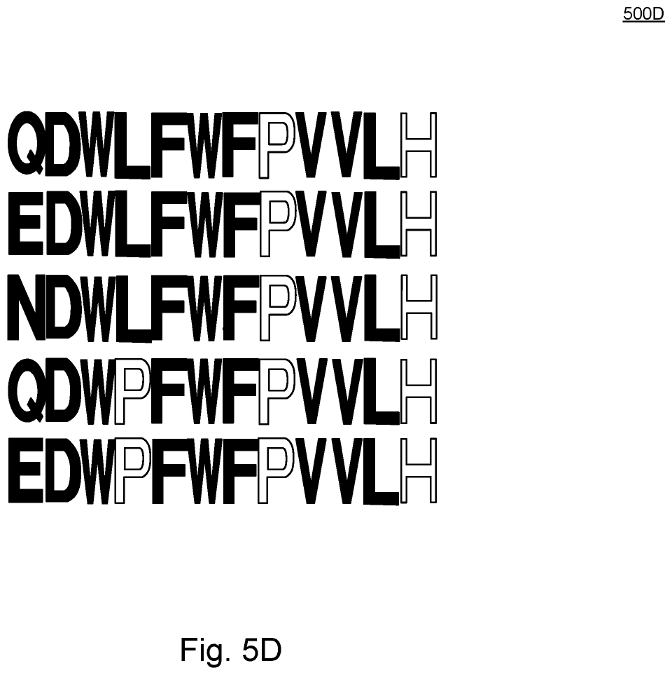

FIG. 5A to FIG. 5D are diagrams depicting an example of enzyme design using a maximum entropy model for the catalytic center of haloalkane dehalogenase according to some implementations.

FIG. 5A shows a distribution 500A of E_{MaxEnt} of the designed sequences in which the E_{MaxEnt} is shifted or adjusted by a constant so that the wild-type (WT) has an energy value of zero. In some embodiments, an enzyme design system (e.g., system 100, model manager 110, training engine 114) may perform parameterization of the MaxEnt model and then redesign the catalytic center of haloalkane dehalogenase based on the statistical energy obtained based on the MaxEnt model. As shown in FIG. 5A, 37% of the designed sequences have lower E_{MaXEnt} than the WT, suggesting possible enhanced catalysis (or enhanced enzyme efficiency).

FIG. 5B shows a sequence logo 500B for the natural MSA. FIG. 5C shows a sequence logo 500C for the WT haloalkane dehalogenase from Xanthobacter autotrophicus. FIG. 5D shows sequence logos 500D for the top 5 designs obtained from the MaxEnt model (e.g., enzyme designs with top-5 lowest statistical energies). For all the sequence logos, the positions from left to right are E56, D124, W125, F128, F172, W175, F222, P223, V226, L262, L263, and H289. Interestingly, one of the top five designs is a consensus design where the residue is replaced by the most frequently observed amino acid in the natural MSA (see FIG. 5C). Consensus design has already been shown effective in protein engineering; and it turns out to be a special case of the MaxEnt model where epistasis is considered.

Embodiments of analysis of protein structure will be described in more detail. In some embodiments, an enzyme design system (e.g., system 100, enzyme data manager 104 in FIG. 1) may analyze, determine, identify (based on enzyme data, e.g., enzyme data 108 in FIG. 1) a distance from mutated residues to a substrate. The PDB IDs used by the enzyme design system according to some embodiments are summarized in Table S10 below. In some embodiments, enzyme design system (e.g., system 100, enzyme data manager 104 in FIG. 1) may use a software package or library (e.g., the Biopython package) in the analysis of the distance. For alcohol dehydrogenase, the co-factor NADP⁺ and catalytic triad may be used as the reference point because of the absence of substrate.

Embodiments of generating and processing MSA will be described in more detail. In some embodiments, for a protein family with a given target sequence, an enzyme design system (e.g., system 100, or enzyme data manager 104) may generate MSA by a profile hidden Markov model (HMM) homology searching tool (e.g., jackhammer) against a database (e.g., the UniRef90 database (release 2021_03)) with five search iterations. In some embodiments, the enzyme design system may adopt a default threshold of 0.7 for the length-normalized bit scores, and may slightly change or adapt the bit score to ensure sufficiently evolutionarily-related sequences. In some embodiments, for ketosteroid Isomerase, the enzyme design system may adopt a smaller bit-score of 0.5 to provide enough sequences. In some embodiments, for alcohol dehydrogenase and trypsin, the enzyme design system may use a bit-score of 1.15. In some embodiments, for beta-lactamase, the enzyme design system may obtain the MSA from an external source. In some embodiments, the enzyme design system may set, determine, or adjust the ratio between sequence number and amino acid number to be greater than 8 for every protein family. The enzyme design system may generate, collect, or obtain enough evolutionarily-related sequences to provide a good MSA statistic.

In some embodiments, after generating, collecting or obtaining the MSA, the enzyme design system may then process the MSA by excluding the sites with more than 30% gaps. The enzyme design system may quantify the similarity between any two sequences by hamming distance, and each sequence may be assigned a weight of 1/(number of sequences>80% identity) to down-weight redundant sequences. Afterward, the enzyme design system may calculate, determine, or generate the statistics (<Sᵢ>ₑₓₚ, <SᵢSⱼ>ₑₓₚ) to constrain the MaxEnt model.

The summary of the MSA processed by the enzyme design system according to implementations is shown in Table S11. In this example, the summary contains an enzyme Commission (EC) number, a UniProt (or PDB) ID of the target sequence, a sequence number, and a sequence number/Residue number (after filtering out gap regions).

Embodiments of a pseudo-likelihood (PLL) approximation and results therefrom will be described in more detail. In some embodiments, an enzyme design system (e.g., system 100, model manager 110) may obtain, calculate or determine the correlation values from the parameterization using the PLL approximation (see Table S12 below). It is found that the correlations using the PLL approximation (see Table S12 below) are consistent with the correlations using a rigorous sampling; and that such a consistent result again validates the correlation-related findings.

Embodiments of an independent model and results therefrom will be described in more detail. In some embodiments, an enzyme design system (e.g., system 100, model manager 110) may obtain, calculate or determine the correlation values from an independent model for which each site is considered independently. For example, the MaxEnt Hamiltonian is given by the following equation:

$${E}_{i n d e p e n d e n t} ( S ) = {\sum }_{i} {h}_{i} {S}_{i} , where {h}_{i} = ln ( {〈 {s}_{i} 〉}_{e x p} + 1 {0}^{- 4} ) . \qquad ( Equation 9 )$$

In Equation 9, the smaller value of 10-4 is introduced to avoid the zero frequency of unseen amino acids. The correlations using the independent model are listed in Table S13 below. It is noted that the MaxEnt model considering pair-wise epistasis is much better than the independent model. In particular, the independent model for alcohol dehydrogenase shows an opposite correlation to the MaxEnt model. There are many higher-order mutants (e.g., up to the tenth order) compared with other enzymes. Such a result might be the most substantial evidence to show the importance of considering epistasis for mutation effects.

Now, embodiments of enzyme design using the MaxEnt model to improve enzyme efficiency will be described in more detail. In some embodiments, with the parameterized MaxEnt model, an enzyme design system (e.g., system 100, model manager 110) may sample the Hamiltonian to generate new sequences. The enzyme design system (e.g., system 100, enzyme data manager 104, mutant selection manager 106, model manager 110) may redesign the catalytic center based on the finding of a systematically significant correlation between E_{MaxEnt} and enzyme catalysis. In some embodiments, for haloalkane dehalogenase, the enzyme design system may use the X-ray structure of haloalkane dehalogenase from Xanthobacter autotrophicus (PDB ID: 2dhc) to select active site residues. Here, residues may be considered to be within a distance of 5.0 Å from the substrate 1,2-dichloroethane (e.g., in or at the catalytic center). In total, the enzyme design system may identify 13 residues, including E56, D124 (general base), W125, F128, F164, F172, W175, F222, P223, V226, L262, L263, and H289. The enzyme design system may then remove the site 164 during the post-processing of MSA because the gap ratio is larger than the threshold, so as to obtain 12 sites to sample. Here, 2dhc numbering is used throughout the present disclosure for haloalkane dehalogenase.

In some embodiments, an enzyme design system (e.g., system 100, enzyme data manager 104, mutant selection manager 106, model manager 110) may use a Monte Carlo step of 10000 with a recording frequency of 1. The enzyme design system may use multiple processors (e.g., 16 processors) to produce 160000 sequences, among which 7458 sequences are unique and 2760 sequences have lower statistical energies (e.g., E_{MaxEnt}) than a statistical energy of the wild-type (WT).

Now, results using the MaxEnt model according to some embodiments will be described in more detail in Table Sᵢ to Table S12.

TABLE S1
Experimental data for haloalkane dehalogenase

| | k_{cat} | Km | k_{cat}/Km |
|---|---|---|---|
| mutant | (s⁻¹) | (mM) | (s⁻¹M⁻¹) |
| WT | 3.3 | 0.53 | 6.2E+03 |
| W125F | 1.11 | 5.1 | 2.06E+02 |
| F172Y | 1.5 | 4.96 | 3.0E+02 |
| F172W | 2.9 | 5.13 | 5.6E+02 |
| W175Y | 0.33 | 2.85 | 1.0E+00 |
| V226A | 3.8 | 1.5 | 2.5E+03 |
| W125F/V226Q | 0 | | 0 |
| W125Q | 0.0875 | 12.5 | 7.E+00 |
| W175Q | | | 5.6E−02 |

The experimental data shown in Table S1 are obtained based on (1) UniProt_ID: P22643; (2) Substrate: 1,2-dichloroethane; (3) The non-enzymatic mutants were assigned a relatively small k_{cat} value which is 0.01 during log transformation; (4) references: WT, F172Y, F172W, V226A; W175Y; W125F/V226Q; (5) except for the W125F/V226Q, which does not show enzymatic function measured with pH 8.6 and temperature of 37° C., the other mutants are measured with the same experimental condition (pH 8.2, 30° C.).

TABLE S2
Experimental data for chorismate mutase

| | k_{cat} | Km | k_{cat}/Km | Tm |
|---|---|---|---|---|
| mutant | (s⁻¹) | (μM) | (s⁻¹M⁻¹) | (° C.) |
| WT | 39 | 300 | 1.3E+05 | 61.4 |
| L7C | | | 1.0E+04 | 52.0 |
| L7I | 36 | 300 | 1.2E+05 | 65.8 |
| L7M | | | 4.4E+04 | 60.3 |
| L7F | 48 | 350 | 1.4E+05 | 62.3 |
| L7T | | | 1.3E+04 | 59.2 |
| L7V | 36 | 700 | 5.0E+04 | 61.9 |
| D48N | | | 7.9E+03 | 56.5 |
| D48C | | | 1.8E+04 | 53.8 |
| D48Q | | | 9.8E+03 | 55.5 |
| D48L | | | 7.1E+03 | 57.7 |
| A32S | 45 | 220 | 2.1E+05 | 61.7 |
| A32T | 61 | 380 | 1.6E+05 | 62.8 |
| I81L | | | 5.5E+04 | 57.7 |
| I81M | 27 | 270 | 1.0E+05 | 64.6 |
| I81F | | | 8.7E+03 | 59.0 |
| V35A | 20 | 520 | 3.9E+04 | 61.2 |
| V35C | 18.6 | 360 | 5.2E+04 | 61.8 |
| V35I | 65 | 440 | 1.5E+05 | 66.6 |
| V35L | | | 1.3E+04 | 66.7 |
| V35M | | | 5.2E+04 | 68.9 |
| V35T | | | 2.0E+04 | 56.5 |
| V85A | | | 1.2E+04 | 62.7 |
| V85R | | | 4.2E+04 | 66.9 |
| V85N | | | 1.2E+04 | 66.8 |
| V85C | 32 | 570 | 5.4E+04 | 54.7 |
| V85I | 55 | 550 | 1.0E+05 | 62.9 |
| V85L | | | 4.3E+04 | 62.2 |
| V85K | | | 1.3E+04 | 67.1 |
| V85M | | | 1.5E+04 | 65.6 |
| V85F | | | 2.1E+04 | 61.7 |
| V85T | | | 2.2E+04 | 62.1 |
| V85Y | | | 2.6E+04 | 65.7 |

The experimental data shown in Table S2 are obtained based on (1) UniProt_ID: POA9J8; (2) Substrate: chorismite.

TABLE S3
Experimental data for alcohol dehydrogenase

| | k_{obs} | Tm |
|---|---|---|
| mutant | (U/mg) | (° C.) |
| WT | 0.6 | 43.00 |
| L150F | 0.5 | 57.25 |
| G149A | 0.4 | 52.50 |
| M157K | 0.4 | 51.00 |
| S197E | 0.5 | 49.00 |
| V238L | 0.4 | 50.00 |
| G159A | 1.2 | 48.50 |
| N212R | 1.1 | 48.00 |
| T194V | | 47.00 |
| V193L | | 46.50 |
| V217P | | 46.00 |
| L150F/M157K | 0.4 | 64.00 |
| L150F/M157K/S197E | 0.1 | 69.00 |
| L150F/M157K/S197E/V238L | 0.2 | 72.00 |
| L150F/M157K/S197E/V238L/N212R | 0.2 | 75.50 |
| L150F/M157K/S197E/V238L/N212R/G149A | 0.1 | 81.00 |
| L150F/M157K/S197E/V238L/N212R/G149A/G159A | 0.1 | 85.00 |
| L150F/M157K/S197E/V238L/N212R/G149A/G159A/V193L/T194V | 0.05 | 90.00 |
| L150F/M157K/S197E/V238L/N212R/G149A/G159A/V193L/T194V/ | 0.03 | 94.50 |
| V217P | | |
| L150F/M157K/V238L/N212R/G149A/G159A/V193L/T194V/V217P | 0.8 | 88.00 |

The experimental data shown in Table S3 are obtained based on (1) PDB_ID: 6TQ3; (2) Substrate: cyclohexanol.

TABLE S4
Experimental data for triosephosphate isomerase

| mutant | k_{cat} (s⁻¹) | k_{cat}/Kₘ (s⁻¹M⁻¹) |
|---|---|---|
| WT | 300 | 4.3E+05 |
| I170A | 17 | 4.6E+03 |
| L230A | 4.7 | 6.1E+04 |
| I170A/L230A | 1.1 | 1.8E+04 |
| E97A | 370 | 2.1E+05 |
| E97D | 380 | 1.7E+05 |
| E97Q | 300 | 7.4E+04 |
| P166A | 27 | 2.5E+05 |

The experimental data shown in Table S4 are obtained based on (1) UniProt_ID: P00942; (2) Substrate: DHAP.

TABLE S5
Experimental data for ketosteroid isomerase

| | Substrate: 5(10)- | | | Substrate: 5- | | |
|---|---|---|---|---|---|---|
| | estrene-3,17-dione | | | androstenedione | | |
| | k_{cat} | Km | k_{cat}/Km | k_{cat} | Km | k_{cat}/Km |
| Mutant | (s⁻¹) | (μM) | (s⁻¹M⁻¹) | (s⁻¹) | (μM) | (s⁻¹M⁻¹) |
| WT | 9.7 | 29 | 3.3E+05 | 1.1E+04 | 81 | 1.4E+08 |
| Y16G | 1.45E−02 | 19 | 7.8E+02 | — | — | — |
| D103L | 5.9E−02 | 39 | 1.5E+03 | — | — | — |
| D103A | 6.6E−02 | 52 | 1.3E+03 | — | — | — |
| D103G | 3.1E−01 | 51 | 6.1E+03 | — | — | — |
| Y16F/D103L | 8.E−06 | 25 | 3.2E−01 | 2.3E−03 | 13 | 1.7E+02 |
| Y16A/D103A | 1.1E−04 | 32 | 3.4E+00 | 2.9E−01 | 27 | 1.1E+03 |
| Y16G/D103G | 1.3E−03 | 36 | 3.9E+01 | 1.2E+01 | 89 | 1.4E+05 |
| Y16G/Y57A/D103G | — | — | — | 7.E−04 | 9.E+01 | 9.E+00 |
| Y16G/Y32F/Y57A/D103G | — | — | — | 5.2E−03 | 67 | 7.8E+01 |
| Y16G/I28G/Y32F/Y57A/ | — | — | — | 5.5E−05 | 140 | 3.9E−01 |
| D103G/M105A/M116G/A118G | | | | | | |

The experimental data shown in Table S5 are obtained based on (1) UniProt_ID: P07445.

TABLE S6
Experimental data for dihydrofolate reductase (Chemical Reviews)

| | | mutation |
|---|---|---|
| mutant | k_{cat} (s⁻¹) | region |
| WT | 12 | |
| T113V | 32.9 | enzyme surface |
| D122N | 4.4 | enzyme surface |
| D122S | 3.3 | enzyme surface |
| D122A | 2.5 | enzyme surface |
| S148D | 4.6 | enzyme surface |
| S148A | 6.6 | enzyme surface |
| S148K | 5.7 | enzyme surface |
| H149Q | 15.1 | enzyme surface |
| F153S | 22.8 | enzyme surface |
| I155N | 23.5 | enzyme surface |
| G121- | 0.43 | enzyme surface |
| Q146-/N147-/S148- | 206 | enzyme surface |
| I14A | 15 | catalytic center |
| W22F | 22 | catalytic center |
| W22H | 1.9 | catalytic center |
| D27S | 0.41 | catalytic center |
| D27E | 41 | catalytic center |
| D27C | 2.2 | catalytic center |
| L28Y | 15 | catalytic center |
| F31Y | 30 | catalytic center |
| F31V | 26 | catalytic center |
| S49A | 9.7 | catalytic center |
| L54F | 6.3 | catalytic center |
| Y100G | 2.9 | catalytic center |
| Y100I | 3.8 | catalytic center |
| M16-/E17-/N18-/A19- | 0.7 | catalytic center |

The experimental data shown in Table S6 are obtained based on (1) UniProt_ID: P0ABQ4; (2) Substrate: NADPH.

TABLE S7
Experimental data for dihydrofolate reductase (PNAS)

| | Tm | k_{cat} | k_{cat}/Km |
|---|---|---|---|
| mutant | (° C.) | (s⁻¹) | (s⁻¹μM⁻¹) |
| WT | 51.7 | 11.65 | 3.6 |
| V40A | 43.2 | 24.05 | 3.22 |
| I61V | 53.4 | 8.99 | 3.7 |
| V75H | 40.6 | 15.5 | 4.57 |
| V75I | 39.2 | 18.25 | 5.51 |
| I91V | 49.1 | 17.87 | 5.13 |
| I91L | 41.4 | | |
| L112V | 47.4 | 11.45 | 2.34 |
| W133F | 46.3 | 13.45 | 6.79 |
| I155T | 43.4 | 11.27 | 2.72 |
| I155L | 45.8 | 12.9 | 5.12 |
| I155A | 38.5 | 11.95 | 2.71 |
| I115V | 51.4 | 10.26 | 2.08 |
| I115A | 46.1 | 7.59 | 0.5 |
| V88I | 44.3 | 13.8 | 3.12 |
| A145T | 51.3 | 10 | 3.6 |

The experimental data shown in Table S7 are obtained based on (1) UniProt_ID: P0ABQ4; (2) Substrate: NADPH.

TABLE S8
Experimental data for beta-lactamase

| | | Substrate: FAP | | | Substrate: AMP | | |
|---|---|---|---|---|---|---|---|
| | | K_{cat} | Km | K_{cat}/Km | K_{cat} | Km | K_{cat}/Km |
| mutant | Tm(° C.) | (s⁻¹) | (μM) | (s⁻¹M⁻¹) | (s⁻¹) | (μM) | (s⁻¹M⁻¹) |
| WT | 51.5 | 1210 | 29.0 | 4.18E+07 | 1737 | 45 | 39 |
| A182V | 58.1 | | | | 1928 | 38 | 50 |
| D177G | 42.2 | 4.27 | 22.0 | 1.94E+05 | | | |
| D177N | 46.7 | 35.0 | 10.2 | 3.44E+06 | | | |
| E102K | 51.0 | 499 | 19.5 | 2.56E+07 | | | |
| E102K/G236S | 46.3 | 8.08 | 5.11 | 1.58E+06 | | | |
| E102K/M180T/ | 55.6 | 19.9 | 2.60 | 7.67E+06 | | | |
| G236S | | | | | | | |
| E102K/R162S | 50.9 | 17.7 | 11.6 | 1.53E+06 | | | |
| E102K/R162S/ | 57.8 | 22.0 | 12.3 | 1.79E+06 | | | |
| M180T | | | | | | | |
| E145G | 56.8 | | | | | | |
| E46L | 55.3 | | | | 1436 | 44 | 33 |
| F58Y | 57.5 | | | | 1845 | 46 | 40 |
| G236A | 47.4 | 187 | 18.3 | 1.02E+07 | | | |
| G236S | 47.0 | 27.8 | 4.15 | 6.70E+06 | | | |
| G76A | 56.4 | | | | 2114 | 61 | 34 |
| G90D | 59.0 | | | | 2519 | 51 | 50 |
| H151R | 57.5 | | | | | | |
| I206M | 57.8 | | | | 1828 | 56 | 33 |
| L199P | 55.6 | | | | | | |
| M180T | 57.7 | 909 | 20.5 | 4.44E+07 | 1566 | 47 | 33 |
| M180T/G236S | 54.0 | 10.8 | 4.83 | 2.23E+06 | | | |
| N272D | 58.0 | | | | 2363 | 104 | 23 |
| R118G | 56.0 | | | | | | |
| R118G/M180T | 61.5 | | | | | | |
| R162H | 48.9 | 132 | 10.2 | 1.30E+07 | | | |
| R162S | 49.8 | 26.1 | 12.1 | 2.16E+06 | | | |
| R271Q | 58.7 | | | | 1614 | 63 | 26 |
| S80H | 57.1 | | | | 1473 | 37 | 40 |
| T261M | 58.3 | | | | 1344 | 60 | 22 |
| V29R | 58.2 | | | | 1428 | 43 | 33 |

The experimental data shown in Table S8 are obtained based on (1) UniProt_ID. P62593.

TABLE S9
Experimental data for trypsin

| | Tm | k_{cat} | k_{cat}/Km |
|---|---|---|---|
| mutant | (K) | (s⁻¹) | (s⁻¹M⁻¹) |
| WT | 325.58 | 27 | 4.1E+05 |
| M109A | 310.65 | 14 | 2.1E+05 |
| L110A | 315.1 | 38 | 2.7E+05 |
| Q211A | 319.85 | 43 | 3.4E+05 |
| T230A | 317.55 | 45 | 4.1E+05 |
| P129A | 318.58 | 40 | 4.0E+05 |
| C160A | 310.02 | 60 | 6.9E+05 |
| M109A/L110A | 310.75 | 18 | 7.4E+05 |
| M109A/Q211A | 306.6 | 35 | 5.6E+05 |
| M109A/T230A | 306.92 | 50 | 6.1E+05 |
| L110A/Q211A | 310.23 | 24 | 1.7E+05 |
| L110A/T230A | 310.12 | 37 | 2.7E+05 |
| Q211A/T230A | 311.32 | 65 | 3.8E+05 |
| G217A | 324.15 | 72 | 9.1E+03 |
| G227A | 326.52 | 4 | 4.8E+02 |
| C196A | 322.05 | 1 | 1.6E+02 |
| D194A | 324.12 | | 1.1E+01 |
| V186A | 324.85 | 23 | 2.8E+05 |
| G217A/Q211A | 319.32 | 51 | 6.2E+03 |
| G217A/C160A | 309.02 | 16 | 3.5E+03 |
| Y37A | 321.22 | 47 | 5.2E+05 |
| Q38A | 325.38 | 39 | 3.7E+05 |
| K231A | 321.75 | 45 | 2.6E+05 |

The experimental data shown in Table S9 are obtained based on (1) UniProt TD: P00763; (2) Substrate: Suc-AAPK-pNA.

TABLE S10
Summary of MSA information

| | | | | Sequence |
|---|---|---|---|---|
| | | Target | Sequence | number/ |
| | EC number | sequence | number | Residue number |
| Haloalkane | EC 3.8.1.5 | P22643/1-310 | 2348 | 8.2 |
| dehalogenase | | | | |
| Chorismate mutase | EC 5.4.99.5 | P0A9J8/1-93 | 11555 | 135.9 |
| Alcohol | EC 1.1.1.1 | 6TQ3/1-246 | 20000 | 85.8 |
| dehydrogenase | | | | |
| Triosephosphate | EC 5.3.1.1 | P00942/1-248 | 22918 | 94.7 |
| isomerase | | | | |
| Dihydrofolate | EC 1.5.1.3 | P0ABQ4/1-159 | 19441 | 125.4 |
| reductase | | | | |
| Beta-lactamase | EC 3.5.2.6 | P62593/24-286 | 8403 | 33.2 |
| Ketosteroid | EC 5.3.3.1 | P07445/1-131 | 2408 | 20.4 |
| isomerase | | | | |
| Trypsin | EC 3.4.21.4 | P00763/24-246 | 20000 | 92.6 |

TABLE S11
Summary of PDB ID

| Enzyme | PDB ID |
|---|---|
| Haloalkane dehalogenase | 2dhc |
| Chorismate mutase | 1ecm |
| Alcohol dehydrogenase | 6tq5 |
| Triosephosphate | 1ney |
| isomerase | |
| Ketosteroid isomerase | 6c17 |
| Dihydrofolate reductase | 1ddr |
| Beta-lactamase | 1bt5 |
| Trypsin | 3tgi |

TABLE S12
Correlations obtained from the PLL approximation

| | E_{MaxEnt} − | E_{MaxEnt} − | E_{MaxEnt} − | Distance to | Mutation | |
|---|---|---|---|---|---|---|
| Enzyme | log k_{cat}/K_{M} | log k_{cat} | Tₘ | substrate(Å) | region | Substrate |
| Haloalkane | −0.86 | −0.94 | | 3.4 ± 0.4 | catalytic | 1,2-dichloroethane |
| dehalogenase | | | | | center | |
| Chorismate | −0.63 | −0.55 | −0.20 | 3.7 ± 0.8 | catalytic | Chorismate |
| mutase | | | | | center | |
| Alcohol | −0.73 | | 0.90 | 6.2 ± 3.8 | catalytic | Cyclohexanol |
| dehydrogenase | | | | | center | |
| Triosephosphate | −0.64 | −0.65 | | 3.9 ± 1.6 | catalytic | DHAP |
| isomerase | | | | | center | |
| Ketosteroid | −0.71 | −0.70 | | 2.1 ± 0.5 | catalytic | 5(10)-estrene-3,17- |
| isomerase | | | | | center | dione |
| | −0.79 | −0.74 | | 3.4 ± 1.3 | catalytic | 5-androstenedione |
| | | | | | center | |
| Dihydrofolate | | −0.74 | | 5.4 ± 2.3 | catalytic | NADPH |
| reductase | | | | | center | |
| | | 0.10 | | 11.4 ± 3.1 | enzyme | |
| | | | | | surface | |
| Dihydrofolate | −0.43 | 0.17 | −0.66 | 10.6 ± 3.8 | enzyme | NADPH |
| reductase | | | | | surface | |
| Beta-lactamase | −0.75 | −0.74 | −0.61 | 10.4 ± 5.8 | enzyme | 6- |
| | | | | | surface | furylacrylpenicillanic |
| | | | | | | acid |
| | −0.49 | 0.00 | | 16.4 ± 4.8 | | ampicillin |
| Trypsin | 0.01 | −0.04 | −0.61 | 10.1 ± 5.2 | enzyme | Suc-AAPK-pNA |
| | | | | | surface | |

It is noted that the order of enzymes are the same as Table 1.

TABLE S13
Correlations obtained from the independent model

| | E_{MaxEnt} − | E_{MaxEnt} − | E_{MaxEnt} − | Distance to | Mutation | |
|---|---|---|---|---|---|---|
| Enzyme | log k_{cat}/K_{M} | log k_{cat} | Tₘ | substrate(Å) | region | Substrate |
| Haloalkane | −0.85 | −0.95 | | 3.4 ± 0.4 | catalytic | 1,2-dichloroethane |
| dehalogenase | | | | | center | |
| Chorismate | −0.41 | −0.44 | −0.32 | 3.7 ± 0.8 | catalytic | Chorismate |
| mutase | | | | | center | |
| Alcohol | 0.60 | | −0.82 | 6.2 ± 3.8 | catalytic | Cyclohexanol |
| dehydrogenase | | | | | center | |
| Triosephosphate | −0.62 | −0.66 | | 3.9 ± 1.6 | catalytic | DHAP |
| isomerase | | | | | center | |
| Ketosteroid | −0.56 | −0.57 | | 2.1 ± 0.5 | catalytic | 5(10)-estrene-3,17- |
| isomerase | | | | | center | dione |
| | −0.76 | −0.72 | | 3.4 ± 1.3 | catalytic | 5-androstenedione |
| | | | | | center | |
| Dihydrofolate | | −0.67 | | 5.4 ± 2.3 | catalytic | NADPH |
| reductase | | | | | center | |
| | | −0.22 | | 11.4 ± 3.1 | enzyme | |
| | | | | | surface | |
| Dihydrofolate | −0.46 | 0.20 | −0.52 | 10.6 ± 3.8 | enzyme | NADPH |
| reductase | | | | | surface | |
| Beta-lactamase | −0.35 | −0.23 | −0.52 | 10.4 ± 5.8 | enzyme | 6- |
| | | | | | surface | furylacrylpenicillanic |
| | | | | | | acid |
| | −0.25 | −0.03 | | 16.4 ± 4.8 | | ampicillin |
| Trypsin | −0.35 | −0.20 | 0.11 | 10.1 ± 5.2 | enzyme | Suc-AAPK-pNA |
| | | | | | surface | |

It is noted that the order of enzymes are the same as Table 1.

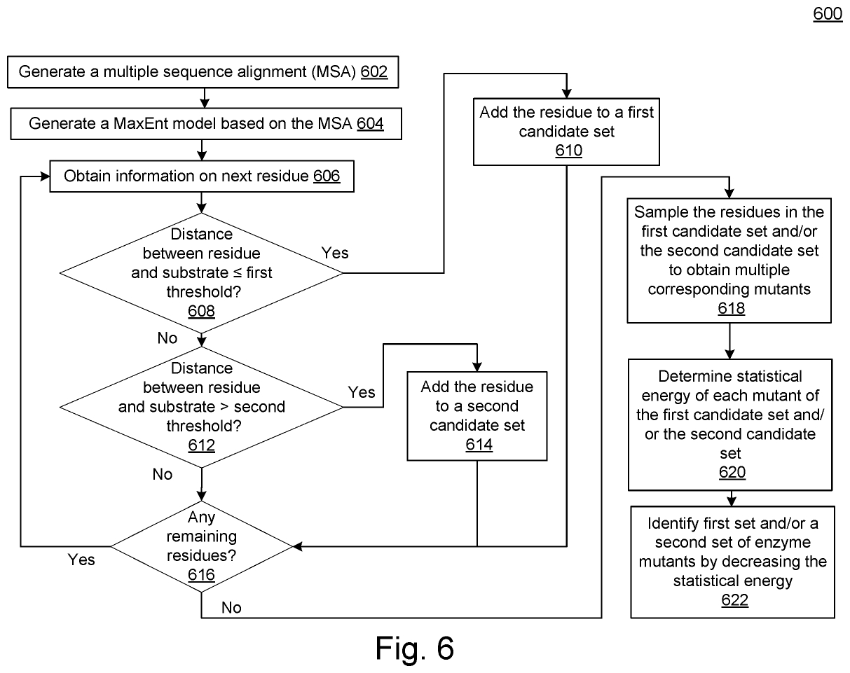

FIG. 6 is a flowchart illustrating an example methodology for identifying enzyme mutants with improved efficiency and/or improved stability according to some implementations. The method includes generating a multiple sequence alignment (MSA) (step 602). The method can include generating a maximum entropy (MaxEnt) model based on the generated MSA (step 604). The method can include obtaining information on a next residue of each mutant of a plurality of enzyme mutants (step 606). The method can include determining whether a distance between a mutated amino acid residue of the mutant and a substrate when the substrate is bound to the mutant is less than or equal to a first threshold (step 608). The method can include adding, in response to determining that the distance is less than or equal to the first threshold, the residue to a first candidate set of enzyme mutants (step 610). The method can include determining whether a distance between a mutated amino acid residue of the mutant and a substrate when the substrate is bound to the mutant is greater than a second threshold (step 612).

The method can include adding, in response to determining that the distance is greater than the second threshold, the residue to a second candidate set of enzyme mutants (step 614). The method can include determining whether there are any remaining residue of each mutant of the plurality of enzyme mutants (step 616). The method can include sampling residues in the first candidate set and/or the second candidate set to obtain corresponding mutants (step 618). The method can include determining, based on the MaxEnt model, a statistical energy of each mutant of the first candidate set and/or determining, based on the MaxEnt model, a statistical energy of each mutant of the second candidate set (step 620). The method can include identifying a first set of enzyme mutants among the first candidate set, based on the statistical energies of the first candidate set, and/or identifying a second set of enzyme mutants among the second candidate set, based on the statistical energies of the second candidate set (step 622).

In further details of step 602, and in some implementations, an enzyme design system (e.g., system 100, enzyme data manager 104) including one or more processors (e.g., processor 721 in FIGS. 7A and 7B) may generate a MSA (step 602). For example, the enzyme design system can retrieve MSA data from a database in the form of extended strings of wild type sequences, and then align different sequences, and/or homologous sequences from different species 260.

In further details of step 604, and in some implementations, the enzyme design system (e.g., model manager 110, training engine 114) may generate, determine, derive, train, learn, or calculate a maximum entropy (MaxEnt) model (e.g., models represented by Equation 1 to Equation 9) based on the generated MSA. In some embodiments, the enzyme design system may generate the MaxEnt model by performing parameterization of the MaxEnt model (e.g., by sampling of the model, replica exchange MCMC, momentum-assisted SGD, MPI, or PLL approximation). For example, referring to Equation (3), the enzyme design system may learn parameters in the MaxEnt model (see Equation (1)) by minimizing the cross-entropy L(θ) between the sequence distribution in the MSA and the model distribution.

In further details of step 606, and in some implementations, the enzyme design system (e.g., enzyme data manager 104 in FIG. 1) may obtain information on a next residue of each mutant of a plurality of enzyme mutants. For example, the enzyme data manager 104 may obtain next residue information by accessing the enzyme data 108 (see FIG. 1).

In further details of 608, and in some implementations, the enzyme design system (e.g., enzyme data manager 104, mutant selection manager 106 in FIG. 1) may determine whether a distance between a mutated amino acid residue of the mutant (e.g., mutant 412 in FIG. 4A) and a substrate (e.g., 1,2-dichloroethane 410 in FIG. 4A) when the substrate is bound to the mutant is less than or equal to a first threshold (e.g., threshold of 7.0 Å for the catalytic center). For example, the enzyme data manager 104 may obtain data relating to structure of enzymes and mutants (e.g., distance between a mutated amino acid residue of an enzyme mutant and a substrate when the substrate is bound to the mutant) by accessing the enzyme data 108 (see FIG. 1). The enzyme design system may add, in response to determining that the distance is less than or equal to the first threshold, the residue to a first candidate set of enzyme mutants (e.g., candidate set of enzyme mutants with improved efficiency) or otherwise associate the residue or mutant with the first candidate set (e.g. adding an identifier of the residue or mutant to a list for the first candidate set) (step 610).

In further details of step 612, and in some implementations, in response to determining that the distance is not less than or equal to the first threshold, the enzyme design system may determine whether a distance between a mutated amino acid residue of the mutant and the substrate when the substrate is bound to the mutant is greater than a second threshold (e.g., threshold of 9.0 Å for the enzyme surface). The enzyme design system may add, in response to determining that the distance is greater than the second threshold, the residue to a second candidate set of enzyme mutants (e.g., candidate set of enzyme mutants with improved stability) or otherwise associate the residue or mutant with the second candidate set (e.g. adding an identifier of the residue or mutant to a list for the second candidate set) (step 614).

For example, the enzyme data manager 104 can calculate a distance between residue(s) of a mutant and a substrate. The enzyme data manager 104 can access, determine, or obtain protein (enzyme) structure for wild-type (WT), from an online database (e.g., PDB) which stores the position of each residue and the substrate. Then, the enzyme data manager 104 can calculate the distance between each residue and the substrate. If the residue is within the first threshold (e.g., 7.0 Å), this residue can be regarded or determined as active site (or catalytic center). If the residue is beyond the second threshold, this residue can be regarded or determined as the enzyme surface. In this manner, the enzyme data manager 104 can calculate a distance between a residue and a substrate solely based on WT structure without knowing the crystal structure for the designed mutant, and therefore the selection of residues is solely based on WT structure. For example, there is a mutant A32S shown in FIG. 4C and FIG. 4D. This mutation occurs at position 32 in the WT. The enzyme data manager 104 can calculate the distance between the residue at position 32 which is alanine (A) and the substrate. Assuming this distance is 3.0 Å which is smaller than the first threshold, the mutant selection manager 106 can include the residue at position 32 into a first candidate set for improved efficiency.

In further details of step 616, and in some implementations, the enzyme design system (e.g., enzyme data manager 104, mutant selection manager 106 in FIG. 1) may determine whether there are any remaining residues of each mutant of the plurality of enzyme mutants (e.g., additional mutants to analyze or characterize). In response to determining that there are any remaining residues of each mutant of the plurality of enzyme mutants, the enzyme design system may proceed to step 606.

In further details of step 618, and in some implementations, in response to determining that there are no remaining residues in the plurality of enzyme mutants, the enzyme design system (e.g., enzyme data manager 104, mutant selection manager 106 in FIG. 1) may sample residues contained in the first candidate set and/or the second candidate set to obtain corresponding mutants in the first candidate set and/or the second candidate set. For example, the mutant selection manager 106 can sample active sites (catalytic centers) contained in the first candidate set and/or the enzyme surface contained in the second candidate set, while fixing the other residues, and obtain the corresponding mutants in the first candidate set and/or the second candidate set, which are candidates of designed enzymes.

In further details of step 620, and in some implementations, the enzyme design system (e.g., model manager 110, mutant selection manager 106 in FIG. 1) may determine, based on the MaxEnt model (e.g., Equation 1), a statistical energy (e.g., E_{MaxEnt}(S)) of each mutant of the first candidate set (e.g., candidate set of enzyme mutants with improved efficiency). Similarly, the enzyme design system may determine, based on the MaxEnt model, a statistical energy of each mutant of the second candidate set (e.g., candidate set of enzyme mutants with improved stability).

In further details of step 622, and in some implementations, the enzyme design system (e.g., mutant selection manager 106 in FIG. 1) may identify a first set of enzyme mutants (e.g., a set of selected, identified, or redesigned mutants with improved catalytic efficiency) among the first candidate set, based on the statistical energies of the first candidate set. For example, the enzyme design system may identify or select, among the first candidate set, the first set of enzyme mutants with top-5 lowest statistical energies. In some embodiments, the enzyme design system may identify or select, among the first candidate set, the first set of enzyme mutants that have statistical energies less than a predetermined threshold. The enzyme design system may rank or order the calculated energies, and identify, as mutants with improved efficiency, one or more enzyme mutants with their energies lower than an energy of wild-type (WT) enzymes (see FIG. 5C). For example, the mutants with improved efficiency may be top-5 mutant designs shown in FIG. 5D. In other words, the enzyme design system can obtain a set of enzyme mutants with improved efficiency by decreasing E_{MaxEnt}(S) or increasing P_{MaxEnt}(S) with respect to mutations at the catalytic center.

In some embodiments, the enzyme design system (e.g., mutant selection manager 106 in FIG. 1) may identify a second set of enzyme mutants (e.g., a set of selected, identified, or redesigned mutants with improved stability) among the second candidate set, based on the statistical energies of the second candidate set. For example, the enzyme design system may identify or select, among the second candidate set, the second set of enzyme mutants with top-5 lowest statistical energies. In some embodiments, the enzyme design system may identify or select, among the second candidate set, the second set of enzyme mutants that have statistical energies less than a predetermined threshold. The enzyme design system may rank or order the calculated energies, and identify, as mutants with improved stability, one or more enzyme mutants with their energies lower than an energy of wild-type enzymes (see FIG. 5C). In other words, the enzyme design system can obtain a set of enzyme mutants with improved stability by decreasing E_{MaxEnt} (S) or increasing P_{MaxEnt}(S) with respect to mutations at the enzyme surface.

Accordingly, the systems and methods discussed herein provide for enzyme analysis and design with advanced classification and prediction of stability and/or efficiency. According to certain aspects, implementations in the present disclosure relate to a method for producing enzyme mutants, may include generating, by one or more processors (e.g., processor 721 in FIG. 7A and FIG. 7B), a maximum entropy model (e.g., Equation 1 to Equation 9) based on homologous sequences of wild-type enzymes (e.g., MSA or sequence data included in enzyme data 108 in FIG. 1). The method may include obtaining, by the one or more processors, information on a plurality of enzyme mutants (e.g., distance between a mutated amino acid residue of a mutant and a substrate when the substrate is bound to the mutant). The method may include determining, by the one or more processors, whether a distance between a mutated amino acid residue of each mutant of the plurality of enzyme mutants (e.g., mutant 412 in FIG. 4A) and a corresponding substrate (e.g., 1,2-dichloroethane 410 in FIG. 4A) when the substrate is bound to the mutant is less than or equal to a first threshold (e.g., threshold of 7.0 Å for the catalytic center). The method may include determining, based on a result of the determination with the first threshold, a first candidate set of the plurality of enzyme mutants (e.g., candidate set of enzyme mutants with improved efficiency). The method may include determining, by the one or more processors based on the maximum entropy model (e.g., Equation 1), a statistical energy (e.g., E_{MaxEnt}(S)) of each mutant of the first candidate set of enzyme mutants. The method may include identifying, by the one or more processors based on the determined statistical energies, a first set of enzyme mutants (e.g., a set of selected, identified, or redesigned mutants with improved catalytic efficiency) among the first candidate set of enzyme mutants.

In some embodiments, the method may include determining, by the one or more processors, the distance between a mutated amino acid residue of each mutant of a plurality of enzyme mutants and the corresponding substrate when the substrate is bound to the mutant is greater than a second threshold (e.g., threshold of 9.0 Å for the enzyme surface), and determining, based on a result of the determination with the second threshold, a second candidate set of the plurality of enzyme mutants (e.g., candidate set of enzyme mutants with improved stability). The method may include determining, by the one or more processors based on the maximum entropy model (e.g., Equation 1), a statistical energy (e.g., E_{MaxEnt}(S)) of each mutant of the second candidate set of enzyme mutants. The method may include identifying, by the one or more processors based on the determined statistical energies of the second candidate set of enzyme mutants, a second set of enzyme mutants (e.g., a set of selected, identified, or redesigned mutants with improved stability) among the second candidate set of enzyme mutants.

In some embodiments, the method may include producing, based on one of the first set of enzyme mutants (e.g., a set of selected, identified, or redesigned mutants with improved catalytic efficiency; e.g., top-5 mutant designs shown in FIG. 5D), a recombinant enzyme comprising at least one non-naturally occurring amino acid mutation.

In some embodiments, a recombinant enzyme may include at least one non-naturally occurring amino acid mutation, and the recombinant enzyme may be one of the first set of enzyme mutants.

According to certain aspects, implementations in the present disclosure relate to a system (e.g., an enzyme design system 100, one or more components thereof 104, 106, 110, or training engine 114) for producing enzyme mutants, may include one or more processors (e.g., processor 721 in FIG. 7A and FIG. 7B) configured to generate a maximum entropy model (e.g., Equation 1 to Equation 9) based on homologous sequences of wild-type enzymes (e.g., MSA or sequence data included in enzyme data 108 in FIG. 1). The one or more processors may be configured to obtain information on a plurality of enzyme mutants (e.g., distance between a mutated amino acid residue of a mutant and a substrate when the substrate is bound to the mutant), determine whether a distance between a mutated amino acid residue of each mutant of the plurality of enzyme mutants (e.g., mutant 412 in FIG. 4A) and a corresponding substrate (e.g., 1,2-dichloroethane 410 in FIG. 4A) when the substrate is bound to the mutant is less than or equal to a first threshold (e.g., threshold of 7.0 Å for the catalytic center), and determine, based on a result of the determination with the first threshold, a first candidate set of the plurality of enzyme mutants (e.g., candidate set of enzyme mutants with improved efficiency).

The one or more processors may be configured to determine, based on the maximum entropy model (e.g., Equation 1), a statistical energy (e.g., E_{MaxEnt}(S)) of each mutant of the first candidate set of enzyme mutants, and identify, based on the determined statistical energies, a first set of enzyme mutants (e.g., a set of selected, identified, or redesigned mutants with improved catalytic efficiency) among the first candidate set of enzyme mutants (e.g., candidate set of enzyme mutants with improved efficiency).

In some embodiments, the one or more processors may be configured to determine the distance between a mutated amino acid residue of each mutant of a plurality of enzyme mutants and the corresponding substrate when the substrate is bound to the mutant is greater than a second threshold (e.g., threshold of 9.0 Å for the enzyme surface), and determine, based on a result of the determination with the second threshold, a second candidate set of the plurality of enzyme mutants (e.g., candidate set of enzyme mutants with improved stability). The one or more processors may be configured to determine, based on the maximum entropy model (e.g., Equation 1), a statistical energy (e.g., E_{MaxEnt}(S)) of each mutant of the second candidate set of enzyme mutants, and identify, based on the determined statistical energies of the second candidate set of enzyme mutants, a second set of enzyme mutants (e.g., a set of selected, identified, or redesigned mutants with improved stability) among the second candidate set of enzyme mutants.

Having discussed specific embodiments of the present solution, it may be helpful to describe aspects of the operating environment, as well as associated system components (e.g., hardware elements) in connection with the methods and systems described herein.

The systems discussed herein may be deployed as, and/or executed on, any type and form of computing device, such as a computer, network device or appliance capable of communicating on any type and form of network and performing the operations described herein. FIG. 7A and FIG. 7B depict block diagrams of a computing device 700 useful for practicing an embodiment of wireless communication devices or an access point. As shown in FIGS. 7A and 7B, each computing device 700 includes a central processing unit 721, and a main memory unit 722. As shown in FIG. 7A, a computing device 700 may include a storage device 728, an installation device 716, a network interface 718, an I/O controller 723, display devices 724a-724n (generally referred to using reference numeral 724), a keyboard 726 and a pointing device 727, such as a mouse. The storage device 728 may include, without limitation, an operating system and/or software. As shown in FIG. 7B, each computing device 700 may also include additional optional elements, such as a memory port 703, a bridge 770, one or more input/output devices 730a-730n (generally referred to using reference numeral 730), and a cache memory 740 in communication with the central processing unit (CPU) 721.

The central processing unit 721 is any logic circuitry that responds to and processes instructions fetched from the main memory 722. In many embodiments, the central processing unit 721 is provided by a microprocessor unit, such as: those manufactured by Intel Corporation of Mountain View, California; those manufactured by International Business Machines of White Plains, New York; or those manufactured by Advanced Micro Devices of Sunnyvale, California. The computing device 700 may be based on any of these processors, or any other processor capable of operating as described herein.

Main memory 722 may be one or more memory chips capable of storing data and allowing any storage location to be directly accessed by the central processing unit 721, such as any type or variant of Static random access memory (SRAM), Dynamic random access memory (DRAM), Ferroelectric RAM (FRAM), NAND Flash, NOR Flash, and Solid State Drives (SSD). The main memory 722 may be based on any of the above described memory chips, or any other available memory chips capable of operating as described herein. In the embodiment shown in FIG. 7A, the central processing unit 721 communicates with main memory 722 via a system bus 750 (described in more detail below). FIG. 7B depicts an embodiment of a computing device 700 in which the processor communicates directly with main memory 722 via a memory port 703. For example, in FIG. 7B the main memory 722 may be DRDRAM.

FIG. 7B depicts an embodiment in which the CPU processor 721 (also referred to as the main processor 721) communicates directly with cache memory 740 via a secondary bus, sometimes referred to as a backside bus. In other embodiments, the main processor 721 communicates with cache memory 740 using the system bus 750. Cache memory 740 typically has a faster response time than main memory 722 and is provided by, for example, SRAM, BSRAM, or EDRAM. In the embodiment shown in FIG. 7B, the main processor 721 communicates with various I/O devices 730a-n via a local system bus 750. Various buses may be used to connect the central processing unit 721 to any of the I/O devices 730a-n, for example, a VESA VL bus, an ISA bus, an EISA bus, a MicroChannel Architecture (MCA) bus, a PCI bus, a PCI-X bus, a PCI-Express bus, or a NuBus. For embodiments in which the I/O device is a video display 724, the processor 721 may use an Advanced Graphics Port (AGP) to communicate with the display 724. FIG. 7B depicts an embodiment of a computing device 700 in which the main processor 721 may communicate directly with I/O device 730b, for example, via HYPERTRANSPORT, RAPIDIO, or INFINIBAND communications technology. FIG. 7B also depicts an embodiment in which local busses and direct communication are mixed: the main processor 721 communicates with I/O device 730a using a local interconnect bus while communicating with I/O device 730b directly.

A wide variety of I/O devices 730a-730n may be present in the computing device 700. Input devices include keyboards, mice, trackpads, trackballs, microphones, dials, touch pads, touch screens, and drawing tablets. Output devices include video displays, speakers, inkjet printers, laser printers, projectors and dye-sublimation printers. The I/O devices may be controlled by an I/O controller 723 as shown in FIG. 7A. The I/O controller may control one or more I/O devices, such as a keyboard 726 and a pointing device 727, e.g., a mouse or optical pen. Furthermore, an I/O device may also provide storage and/or an installation device 716 for the computing device 700. In still other embodiments, the computing device 700 may provide USB connections (not shown) to receive handheld USB storage devices such as the USB Flash Drive line of devices manufactured by Twintech Industry, Inc. of Los Alamitos, California.

Referring again to FIG. 7A, the computing device 700 may support any suitable installation device 716, such as a disk drive, a CD-ROM drive, a CD-R/RW drive, a DVD-ROM drive, a flash memory drive, tape drives of various formats, USB device, hard-drive, a network interface, or any other device suitable for installing software and programs. The computing device 700 may further include a storage device, such as one or more hard disk drives or redundant arrays of independent disks, for storing an operating system and other related software, and for storing application software programs such as any program or application 720 for implementing (e.g., configured and/or designed for) the systems and methods described herein. Optionally, any of the installation devices 716 could also be used as the storage device. Additionally, the operating system and the software can be run from a bootable medium.

Furthermore, the computing device 700 may include a network interface 718 to interface to a network through a variety of connections including, but not limited to, standard telephone lines, LAN or WAN links (e.g., 802.11, T1, T3, 56kb, X.25, SNA, DECNET), broadband connections (e.g., ISDN, Frame Relay, ATM, Gigabit Ethernet, Ethernet-over-SONET), wireless connections, or some combination of any or all of the above. Connections can be established using a variety of communication protocols (e.g., TCP/IP, IPX, SPX, NetBIOS, Ethernet, ARCNET, SONET, SDH, Fiber Distributed Data Interface (FDDI), RS232, IEEE 802.11, IEEE 802.11a, IEEE 802.11b, IEEE 802.11g, IEEE 802.11n, IEEE 802.11ac, IEEE 802.11ad, CDMA, GSM, WiMax, and direct asynchronous connections). In one embodiment, the computing device 700 communicates with other computing devices 700 via any type and/or form of gateway or tunneling protocol such as Secure Socket Layer (SSL) or Transport Layer Security (TLS). The network interface 718 may include a built-in network adapter, network interface card, PCMCIA network card, card bus network adapter, wireless network adapter, USB network adapter, modem, or any other device suitable for interfacing the computing device 700 to any type of network capable of communication and performing the operations described herein.

In some embodiments, the computing device 700 may include or be connected to one or more display devices 724a-724n. As such, any of the I/O devices 730a-730n and/or the I/O controller 723 may include any type and/or form of suitable hardware, software, or combination of hardware and software to support, enable or provide for the connection and use of the display device(s) 724a-724n by the computing device 700. For example, the computing device 700 may include any type and/or form of video adapter, video card, driver, and/or library to interface, communicate, connect or otherwise use the display device(s) 724a-724n. In one embodiment, a video adapter may include multiple connectors to interface to the display device(s) 724a-724n. In other embodiments, the computing device 700 may include multiple video adapters, with each video adapter connected to the display device(s) 724a-724n. In some embodiments, any portion of the operating system of the computing device 700 may be configured for using multiple displays 724a-724n. In some embodiments, a computing device 700 may be configured to have one or more display devices 724a-724n.

In further embodiments, an I/O device 730 may be a bridge between the system bus 750 and an external communication bus, such as a USB bus, an Apple Desktop Bus, an RS-232 serial connection, a SCSI bus, a FireWire bus, a FireWire 800 bus, an Ethernet bus, an AppleTalk bus, a Gigabit Ethernet bus, an Asynchronous Transfer Mode bus, a FibreChannel bus, a Serial Attached small computer system interface bus, a USB connection, or a HDMI bus.

A computing device 700 of the sort depicted in FIGS. 7A and 7B may operate under the control of an operating system, which controls scheduling of tasks and access to system resources. The computing device 700 can be running any operating system, such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: Android, produced by Google Inc.; WINDOWS 7 and 8, produced by Microsoft Corporation of Redmond, Washington; MAC OS, produced by Apple Computer of Cupertino, California; WebOS, produced by Research In Motion (RIM); OS/2, produced by International Business Machines of Armonk, New York; and Linux, a freely-available operating system distributed by Caldera Corp. of Salt Lake City, Utah, or any type and/or form of a Unix operating system, among others.

The computing device 700 can be any workstation, telephone, desktop computer, laptop or notebook computer, server, handheld computer, mobile telephone or other portable telecommunications device, media playing device, gaming system, mobile computing device, or any other type and/or form of computing, telecommunications or media device that is capable of communication. The computing device 700 has sufficient processor power and memory capacity to perform the operations described herein.

In some embodiments, the computing device 700 may have different processors, operating systems, and input devices consistent with the device. For example, in one embodiment, the computing device 700 is a smart phone, mobile device, tablet or personal digital assistant. In still other embodiments, the computing device 700 is an Android-based mobile device, an iPhone smart phone manufactured by Apple Computer of Cupertino, California, or a Blackberry or WebOS-based handheld device or smart phone, such as the devices manufactured by Research In Motion Limited. Moreover, the computing device 700 can be any workstation, desktop computer, laptop or notebook computer, server, handheld computer, mobile telephone, any other computer, or other form of computing or telecommunications device that is capable of communication and that has sufficient processor power and memory capacity to perform the operations described herein.

Although the disclosure may reference one or more “users”, such “users” may refer to user-associated devices or stations (STAs), for example, consistent with the terms “user” and “multi-user” typically used in the context of a multi-user multiple-input and multiple-output (MU-MIMO) environment.

Although examples of communications systems described above may include devices and APs operating according to an 802.11 standard, it should be understood that embodiments of the systems and methods described can operate according to other standards and use wireless communications devices other than devices configured as devices and APs. For example, multiple-unit communication interfaces associated with cellular networks, satellite communications, vehicle communication networks, and other non-802.11 wireless networks can utilize the systems and methods described herein to achieve improved overall capacity and/or link quality without departing from the scope of the systems and methods described herein.

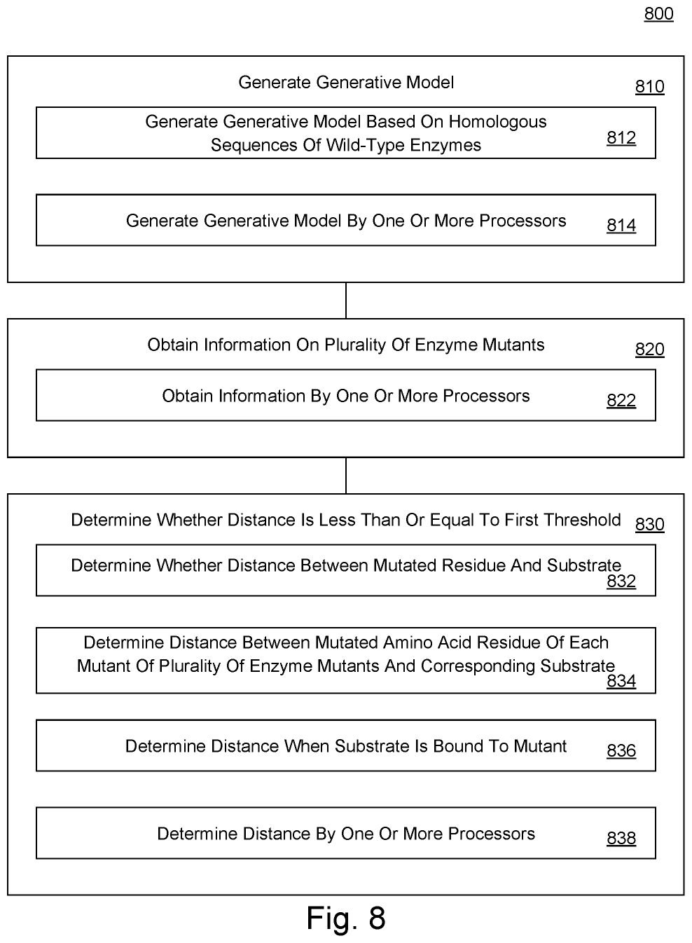

FIG. 8 depicts an example method using a computational architecture for enzyme design according to this disclosure. At least the enzyme design system 100, or any component thereof, can perform method 800. At 810, the method 800 can generate a generative model. For example, the model manager 110 can generate a generative model. For example, the generative model comprises a maximum entropy model, an autoregressive model, a variational autoencoder, a generative adversarial network, a flow-based generative model, or an energy based model. At 812, the method 800 can generate the generative model based on homologous sequences of wild-type enzymes. At 814, the method 800 can generate the generative model by one or more processors. At 820, the method 800 can obtain information on a plurality of enzyme mutants. At 822, the method 800 can obtain the information by the one or more processors. At 830, the method 800 can determine whether a distance is less than or equal to a first threshold. For example, the enzyme data manager 104 can determine whether a distance is less than or equal to a first threshold. At 832, the method 800 can determine whether a distance between a mutated residue and a substrate. At 834, the method 800 can determine the distance between a mutated amino acid residue of each mutant of the plurality of enzyme mutants and a corresponding substrate. At 836, the method 800 can determine the distance when the substrate is bound to the mutant. At 838, the method 800 can determine the distance by the one or more processors.

For example, the method 800 can include determining, by the one or more processors, whether the distance between the mutated amino acid residue of each mutant of the plurality of enzyme mutants and the corresponding substrate when the substrate is bound to the mutant is greater than a second threshold. For example, the enzyme data manager 104 can determine whether the distance between the mutated amino acid residue of each mutant of the plurality of enzyme mutants and the corresponding substrate when the substrate is bound to the mutant is greater than a second threshold. The method 800 can include selecting, based on a result of the determination with the second threshold, a second candidate set of enzyme mutants. The method can include calculating, by the one or more processors based on the generative model, a statistical energy of each mutant of the second candidate set of enzyme mutants. The method can include identifying, by the one or more processors based on the calculated statistical energies of the second candidate set of enzyme mutants, a second set of enzyme mutants among the second candidate set of enzyme mutants, such that the second set of enzyme mutants have higher stability than remaining ones of the second candidate set of enzyme mutants. For example, the method can include where the second threshold is greater than the first threshold.

For example, the method 800 can include determining whether there are any remaining residues of each mutant of the plurality of enzyme mutants. For example, the enzyme data manager 104 can determine whether there are any remaining residues of each mutant of the plurality of enzyme mutants. The method can include, in response to determining that there are no remaining residues in the plurality of enzyme mutants, sampling residues contained in the first candidate set to obtain the corresponding mutants in the first candidate set.

For example, the method 800 can include selecting a second set of enzyme candidates. For example, the mutant selection manager can select a second set of enzyme candidates. For example, the method can include where selecting the second candidate set comprises in response to determining that the distance between the mutated amino acid residue of each mutant of the plurality of enzyme mutants and the corresponding substrate when the substrate is bound to the mutant is greater than the second threshold, adding the mutated amino acid residue to the second candidate set. For example, the method can include where selecting the second set of enzyme mutants comprises identifying, as mutants with improved stability, one or more enzyme mutants with statistical energies thereof lower than a statistical energy of wild-type enzymes. For example, the method can include sampling residues contained in the second candidate set to obtain the corresponding mutants in the second candidate set.

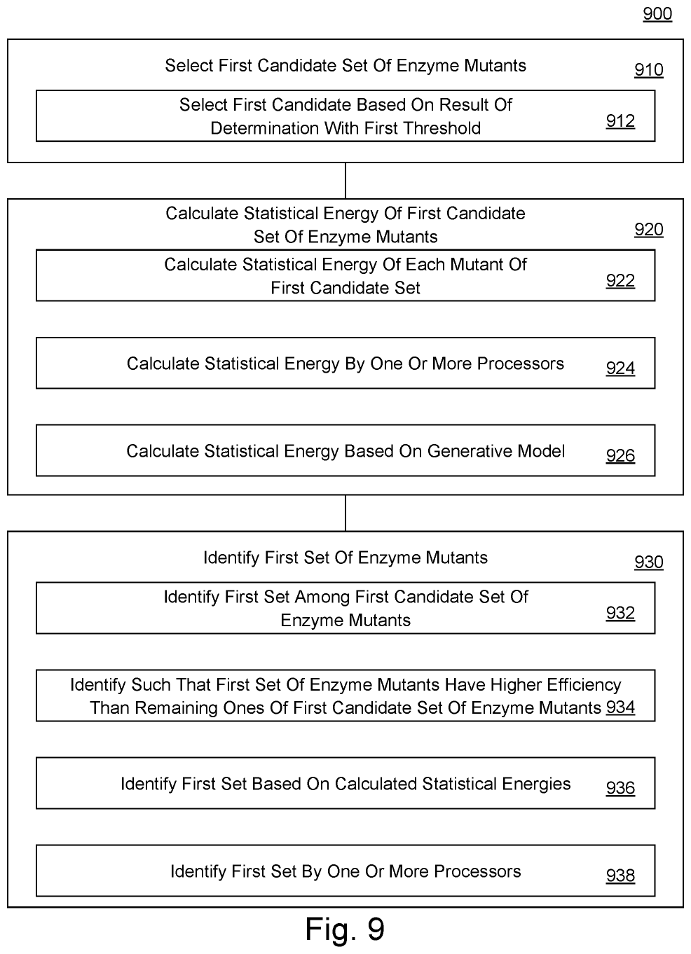

FIG. 9 depicts an example method using a computational architecture for enzyme design according to this disclosure. At least the enzyme design system 100, or any component thereof, can perform method 900. At 910, the method 900 can select a first candidate set of enzyme mutants. For example, the mutant selection manager 106 can select a first candidate set of enzyme mutants. At 912, the method 900 can select the first candidate based on a result of the determination with the first threshold.

At 920, the method 900 can calculate a statistical energy of the first candidate set of enzyme mutants. At 922, the method 900 can calculate the statistical energy of each mutant of the first candidate set. At 924, the method 900 can calculate the statistical energy by the one or more processors. At 926, the method 900 can calculate the statistical energy based on the generative model.

At 930, the method 900 can identify a first set of enzyme mutants. At 932, the method 900 can identify the first set among the first candidate set of enzyme mutants. At 934, the method 900 can identify such that the first set of enzyme mutants have higher efficiency than remaining ones of the first candidate set of enzyme mutants. At 936, the method 900 can identify the first set based on the calculated statistical energies. For example, the determining the first set of enzyme mutants comprises identifying, as mutants with improved efficiency, one or more enzyme mutants with statistical energies lower than a statistical energy of wild-type enzymes. At 938, the method 900 can identify the first set by the one or more processors.

For example, in response to determining that the distance between the mutated amino acid residue of each mutant of the plurality of enzyme mutants and the corresponding substrate when the substrate is bound to the mutant is less than or equal to the first threshold, adding the mutated amino acid residue to the first candidate set. For example, the method can include producing, based on one of the first set of enzyme mutants, a recombinant enzyme that can include at least one non-naturally occurring amino acid mutation.

It should be noted that certain passages of this disclosure may reference terms such as “first” and “second” in connection with devices, modes of operation, transmit chains, antennas, etc., for purposes of identifying or differentiating one from another or from others. These terms are not intended to merely relate entities (e.g., a first device and a second device) temporally or according to a sequence, although in some cases, these entities may include such a relationship. Nor do these terms limit the number of possible entities (e.g., devices) that may operate within a system or environment.

It should be understood that the systems described above may provide multiple ones of any or each of those components and these components may be provided on either a standalone machine or, in some embodiments, on multiple machines in a distributed system. In addition, the systems and methods described above may be provided as one or more computer-readable programs or executable instructions embodied on or in one or more articles of manufacture. The article of manufacture may be a floppy disk, a hard disk, a CD-ROM, a flash memory card, a PROM, a RAM, a ROM, or a magnetic tape. In general, the computer-readable programs may be implemented in any programming language, such as LISP, PERL, C, C++, C#, PROLOG, or in any byte code language such as JAVA. The software programs or executable instructions may be stored on or in one or more articles of manufacture as object code.

While the foregoing written description of the methods and systems enables one of ordinary skill to make and use what is considered presently to be the best mode thereof, those of ordinary skill will understand and appreciate the existence of variations, combinations, and equivalents of the specific embodiment, method, and examples herein. The present methods and systems should therefore not be limited by the above described embodiments, methods, and examples, but by all embodiments and methods within the scope and spirit of the disclosure.

1. A method for producing enzyme mutants, comprising:
generating, by one or more processors, a generative model based on homologous sequences of wild-type enzymes;
obtaining, by the one or more processors, information on a plurality of enzyme mutants;
determining, by the one or more processors, whether a distance between a mutated amino acid residue of each mutant of the plurality of enzyme mutants and a corresponding substrate when the substrate is bound to the mutant is less than or equal to a first threshold;
selecting, based on a result of the determination with the first threshold, a first candidate set of enzyme mutants;
calculating, by the one or more processors based on the generative model, a statistical energy of each mutant of the first candidate set of enzyme mutants; and
identifying, by the one or more processors based on the calculated statistical energies, a first set of enzyme mutants among the first candidate set of enzyme mutants, such that the first set of enzyme mutants have higher efficiency than remaining ones of the first candidate set of enzyme mutants.

2. The method of claim 1, wherein the generative model comprises a maximum entropy model, an autoregressive model, a variational autoencoder, a generative adversarial network, a flow-based generative model, or an energy based model.

3. The method of claim 1, wherein determining the first candidate set comprises:
in response to determining that the distance between the mutated amino acid residue of each mutant of the plurality of enzyme mutants and the corresponding substrate when the substrate is bound to the mutant is less than or equal to the first threshold, adding the mutated amino acid residue to the first candidate set.

4. The method of claim 1, wherein determining the first set of enzyme mutants comprises:
identifying, as mutants with improved efficiency, one or more enzyme mutants with statistical energies lower than a statistical energy of wild-type enzymes.

5. The method of claim 1, further comprising:
determining whether there are any remaining residues of each mutant of the plurality of enzyme mutants; and
in response to determining that there are no remaining residues in the plurality of enzyme mutants, sampling residues contained in the first candidate set to obtain the corresponding mutants in the first candidate set.

6. The method of claim 1, further comprising:
determining, by the one or more processors, whether the distance between the mutated amino acid residue of each mutant of the plurality of enzyme mutants and the corresponding substrate when the substrate is bound to the mutant is greater than a second threshold;
selecting, based on a result of the determination with the second threshold, a second candidate set of enzyme mutants;
calculating, by the one or more processors based on the generative model, a statistical energy of each mutant of the second candidate set of enzyme mutants; and
identifying, by the one or more processors based on the calculated statistical energies of the second candidate set of enzyme mutants, a second set of enzyme mutants among the second candidate set of enzyme mutants, such that the second set of enzyme mutants have higher stability than remaining ones of the second candidate set of enzyme mutants.

7. The method of claim 6, wherein the second threshold is greater than the first threshold.

8. The method of claim 6, wherein selecting the second candidate set comprises:
in response to determining that the distance between the mutated amino acid residue of each mutant of the plurality of enzyme mutants and the corresponding substrate when the substrate is bound to the mutant is greater than the second threshold, adding the mutated amino acid residue to the second candidate set.

9. The method of claim 6, wherein selecting the second set of enzyme mutants comprises:
identifying, as mutants with improved stability, one or more enzyme mutants with statistical energies thereof lower than a statistical energy of wild-type enzymes.

10. The method of claim 6, further comprising:
sampling residues contained in the second candidate set to obtain the corresponding mutants in the second candidate set.

11. The method of claim 1, further comprising:
producing, based on one of the first set of enzyme mutants, a recombinant enzyme comprising at least one non-naturally occurring amino acid mutation.

12. (canceled)

13. A system for producing enzyme mutants, comprising one or more processors in communication with one or more data storage devices storing an enzyme database, a machine learning model, and training instances, the one or more processors configured to:
generate a generative model based on homologous sequences of wild-type enzymes;
obtain information on a plurality of enzyme mutants;
determine whether a distance between a mutated amino acid residue of each mutant of the plurality of enzyme mutants and a corresponding substrate when the substrate is bound to the mutant is less than or equal to a first threshold;
select, based on a result of the determination with the first threshold, a first candidate set of enzyme mutants;
calculate, based on the generative model, a statistical energy of each mutant of the first candidate set of enzyme mutants; and
identify, based on the calculated statistical energies, a first set of enzyme mutants among the first candidate set of enzyme mutants.

14. The system of claim 13, wherein the generative model comprises a maximum entropy model, an autoregressive model, a variational autoencoder, a generative adversarial network, a flow-based generative model, or an energy based model.

15. The system of claim 13, wherein in determining the first candidate set, the one or more processors are configured to:
in response to determining that the distance between the mutated amino acid residue of each mutant of the plurality of enzyme mutants and the corresponding substrate when the substrate is bound to the mutant is less than or equal to the first threshold, add the mutated amino acid residue to the first candidate set.

16. The system of claim 13, wherein in determining the first set of enzyme mutants, the one or more processors are configured to:
identify, as mutants with improved efficiency, one or more enzyme mutants with statistical energies lower than a statistical energy of wild-type enzymes.

17. The system of claim 14, wherein the one or more processors are configured to:
determine whether there are any remaining residues of each mutant of the plurality of enzyme mutants; and
in response to determining that there are no remaining residues in the plurality of enzyme mutants, sample residues contained in the first candidate set to obtain the corresponding mutants in the first candidate set.

18. The system of claim 14, wherein the one or more processors are configured to:
determine whether the distance between the mutated amino acid residue of each mutant of the plurality of enzyme mutants and the corresponding substrate when the substrate is bound to the mutant is greater than a second threshold;
select, based on a result of the determination with the second threshold, a second candidate set of enzyme mutants;
calculate, based on the generative model, a statistical energy of each mutant of the second candidate set of enzyme mutants; and
identify, based on the calculated statistical energies of the second candidate set of enzyme mutants, a second set of enzyme mutants among the second candidate set of enzyme mutants.

19. (canceled)

20. The system of claim 18, wherein in selecting the second candidate set, the one or more processors are configured to:
in response to determining that the distance between the mutated amino acid residue of each mutant of the plurality of enzyme mutants and the corresponding substrate when the substrate is bound to the mutant is greater than the second threshold, add the mutated amino acid residue to the second candidate set.

21. The system of claim 18, wherein in selecting the second set of enzyme mutants, the one or more processors are configured to:
identify, as mutants with improved stability, one or more enzyme mutants with statistical energies thereof lower than a statistical energy of wild-type enzymes.

22. The system of claim 18, wherein the one or more processors are configured to:
sample residues contained in the second candidate set to obtain the corresponding mutants in the second candidate set.
